(19) United States
(12) Patent Application Publication
(10) Pub. No.: US 2016/0262894 A1
(43) Pub. Date: Sep. 15, 2016

(54) TISSUE REPAIR DEVICES AND SCAFFOLDS

(71) Applicants: Brouce N. CRONSTEIN, New York, NY (US); Aranzazu MEDIERO MUNZO, New York, NY (US); John RICCI, Middletown, NJ (US); Stephen ISHACK, New York, NY (US)

(72) Inventors: Bruce N Cronstein, New York, NY (US); Aranzazu Mediero Munoz, New York, NY (US); John Ricci, Middletown, NJ (US); Stephanie Ishack, New York, NY (US)

(21) Appl. No.: 15/032,067

(22) PCT Filed: Oct. 24, 2014
(86) PCT No.: PCT/US14/62088

§ 371 Date: Apr. 26, 2016

Related U.S. Application Data
(60) Provisional application No. 61/896,311, filed on Oct. 28, 2013.

(51) Int. Cl. A61F 2/28; A61L 27/24; A61L 27/54; A61F 2/30; A61L 27/56

(52) U.S. Cl. CPC A61F 2/28; A61F 2/3094; A61L 27/56; A61L 27/54; A61L 27/24; A61F 2002/30011; A61F 2002/30677; A61F 2002/30985; A61L 2300/436; A61L 2300/204; A61L 2430/02

(57) ABSTRACT

The present invention relates to multiphasic, three-dimensionally printed, tissue repair devices or scaffolds useful for promoting bone growth and treating bone fracture, defect or deficiency, methods for making the same and methods for promoting bone growth and treating bone fracture, defect or deficiency using the same. The scaffold has a porous bone ingrowth area containing interconnected struts surrounded by a microporous shell. At the ends of the scaffold, the shell may be extended as a guide flange to stabilize the scaffold between ends of bone. The center of the scaffold may be empty and may serve as a potential marrow space.

### FIELD OF THE INVENTION

The present invention relates to multiphasic, three-dimensionally printed, tissue repair devices or scaffolds having therein or thereon a therapeutically effective amount of an adenosine receptor agonist or an adenosine receptor antagonist, or an agent that upregulates, increases the amount of or increases the biological activity of adenosine or an analog or derivative thereof, and useful for promoting bone growth and treating bone fracture, defect or deficiency, methods for making the same and methods for promoting bone growth and treating bone fracture, defect or deficiency using the same.

### BACKGROUND OF THE INVENTION

Bone and soft tissue defects, in the craniofacial, plastic surgery, and orthopedic arenas are often filled using autogenous tissue grafts, processed human allograft materials, or alloplastic (synthetic) materials, all of which have deficiencies. Autogenous materials must be harvested from another surgical site, and processed human allografts are expensive, inconsistent, and may pose the risk of disease transmission. Alloplastic materials sometimes perform poorly, are sometimes long lasting or permanent, and can become infected. All of these materials have to be shaped to fit complex sites or are granular in form and must somehow be fixed in place. The search continues for a perfect bone repair material—one that can be custom fabricated to fit complex defects, will stimulate bone repair to fill large bone defects, and will eventually dissolve and/or remodel away leaving only regenerated bone. Some alloplastic materials available for similar uses include those described by Owen et al., JBMR Part A 2010, Chen et al. Biomaterials 2011, Kim et al., Tiss Eng Part B, 2010 and Fu et al., Acta Biomaterialia 2011.

Children requiring complex craniofacial repair, like those with alveolar clefts or with Treacher-Collin's syndrome, unlike adults, require fully resorbable materials that can enable bone regeneration in conjunction with craniofacial growth. With bone grafting insufficient to repair these defects, these children require innovation in bone repair technologies. The ideal bone repair scaffold needs to be off-the-shelf and/or custom fabricated to fit closely the lost or missing three dimensional structure. Three dimensional foam scaffold fabrication techniques such as particulate leaching, phase separation/inversion, porogen methods, and spin casting, while controlling overall pore size distribution, do not control individual pore location, pore morphology, and pore interconnectivity; the latter being a well-documented necessity for promoting exchange of nutrients and metabolites as well as promoting conduction of bone and vascular cells through scaffolds (Lee et al., J Mater Sci Mater Med 2010; 21: 3195-3205).

A useful three dimensional printing process, direct write (DW), as detailed by Nadkarni et al., J Am Ceram Soc 2006; 89: 96-103 is based on the extrusion/deposition of colloidal inks as continuous filaments. DW requires minimal processing aids (i.e., polymers) in the ink for self-supporting filament/struts that will enable printing of the lattice structures required for bone scaffolds. The scaffolds are printed by ink extrusion on the XY plane, “writing” the bottom layer, then moving up in Z height to write additional layers until a three dimensional structure is formed. Post-processing of the printed green bodies requires binder burnout and sintering in a high temperature furnace. The resulting scaffolds are of high resolution and very reproducible.

Previous work by Simon et al., J Biomed Mater Res 2007; 83A: 747-758, consisted of filling rabbit calvarial trephine defects of 11 mm with hydroxyapatite (HA). It is possible to increase scaffold resorption by adding, beta-tricalcium-phosphate (β-TCP) to the HA to form a biphasic colloid which has been shown to be osteoconductive and remodelable. Furthermore, calcium sulfate (CS) has been added to fill the space between struts as temporary filler. CS is known to be completely resorbable, osteoconductive, angiogenic, and biocompatible (Thomas et al., J Biomed Mater Res 2009; 88B: 597-610), and in scaffolds serves to act as a filler that dissolves just ahead of the bone ingrowth front.

It would be useful to determine how mesopore space and strut patterns determine the morphology of ingrowing bone. Although many studies have been conducted to investigate the relationship between pore size and bone formation, the optimal pore size is unclear with most studies suggesting a range of 100 to 400 μm (LeGeros, Clin Orthop Relat Res 2002; 395: 81-98). DW allows the production of controlled mesopore sizes in scaffolds. One previous scaffold design for calvaria defects consisted of an 11 mm disk with quadrants comprising different lattice spacings ranging from 250 μm to 400 μm. After 8 and 16 weeks in vivo the smaller-pore regions produced a different pattern of bone growth and scaffold resorption than the larger-pore regions (Ricci et al., J Craniofac Surg 2012; 23: 00-00; Ricci et al., “Biological Mechanisms of Calcium Sulfate Replacement by Bone.” In: Bone Engineering, ed. JE Davies, Em² Inc., Toronto, Ont. Canada, Chapter 30, 332-344, 2000).

The many clinical situations that require extensive complex bone repair and regeneration continue to represent problems without acceptable solutions. The current clinical treatments are compromises that require elaborate and complex autogenous grafting procedures, or they represent imperfect allogeneic or alloplastic treatment options. In all cases these complex bone repair situations require that materials not made for a specific site are fit as well as possible into the defect. It would be desirable to provide new means for printing three dimensional scaffolds composed of osteoconductive biomaterials that have the potential to be custom-fabricated to repair complex defects. Similar tissue repair devices or scaffolds are described by Ricci et al. in PCT/US2013/43336, filed May 30, 2013, the disclosure of which is incorporated herein by reference in its entirety.

Adenosine is a nucleoside that occurs naturally in mammals, which acts as a ubiquitous biochemical messenger. The heart, for instance, produces and releases adenosine in order to modulate heart rate and coronary vasodilation. Likewise, adenosine is produced in the kidney to modulate essential physiological responses, including glomerular filtration rate (GFR), electrolyte reabsorption, and renin secretion.

Adenosine is known to bind to and activate seven-transmembrane spanning G-protein coupled receptors, thereby eliciting a variety of physiological responses. There are 4 known subtypes of adenosine receptors (i.e., A₁, A_{2A}, A_{2B}, and A₃), which mediate different, and sometimes opposing, effects. For example, activation of the adenosine A₁ receptor, elicits an increase in renal vascular resistance, which leads to a decrease in glomerular filtration rate (GFR), while activation of the adenosine A_{2A} receptor elicits a decrease in renal vascular resistance. Conversely, blockade of the A₁ adenosine receptor decreases afferent arteriole pressure, leading to an increase in GFR and urine flow, and sodium excretion. Furthermore, A_{2A} adenosine receptors modulate coronary vasodilation, whereas A_{2B} receptors have been implicated in mast cell activation, asthma, vasodilation, regulation of cell growth, intestinal function, and modulation of neurosecretion (See, Adenosine A_{2B} Receptors as Therapeutic Targets, Drug Dev Res 45:198; Feoktistov et al., Trends Pharmacol Sci 19:148-153 and Ralevic et al., Pharmacological Reviews, 1998; 50: 413-492), and A₃ adenosine receptors modulate cell proliferation processes. Two receptor subtypes (A₁ and A_{2A}) exhibit affinity for adenosine in the nanomolar range while two other known subtypes A_{2B} and A₃ are low-affinity receptors, with affinity for adenosine in the low-micromolar range. A₁ and A₃ adenosine receptor activation can lead to an inhibition of adenylate cyclase activity, while A_{2A} and A_{2B} activation causes a stimulation of adenylate cyclase.

It has been shown that adenosine, acting at specific cell surface receptors, has the potential to suppress inflammation and that inflammation itself may increase extracellular adenosine levels (Cronstein, et al., 1986, Journal of Clinical Investigation 78: 760-770; Cronstein, et al., 1983, Journal of Experimental Medicine 158: 1160-1177). Further, it has been demonstrated that adenosine mediates the anti-inflammatory effects of low-dose methotrexate therapy for Rheumatoid Arthritis (Reviewed in Cronstein, 2005, Pharmacol Rev 57: 163-172). Exploration of the therapeutic and toxic properties of methotrexate in the treatment of RA has led to a number of other potentially important pre-clinical therapeutic developments. Methotrexate increases giant cell formation from peripheral blood monocytes and that this effect is mediated by adenosine acting at A₁ receptors (Merrill, et al., Arth. Rheum. 40: 1308-1315). In addition, A_{2A} receptor antagonists promote giant cell formation by diminishing the effect of endogenous adenosine although the A₁ receptor-mediated promotion of giant cell formation appears to dominate.

A₁ receptor antagonists completely block, in a dose-dependent fashion, osteoclast formation. Similarly, the A₁ receptor antagonists block osteoclast function (resorption of dentin). Six-month old A₁ KO mice demonstrate increased bone density. Their bones demonstrate diminished resorption, and some evidence indicates that the osteoclasts in the A₁ knockout mice do not resorb bone. A murine model of post-menopausal osteoporosis, ovariectomy-induced bone loss, reveals that treatment of mice with an adenosine A₁ receptor antagonist completely prevents ovariectomy-induced bone loss. Adenosine A₁ receptors may be useful in treating and preventing osteoporosis.

Osteoblasts are mononucleate cells that are responsible for bone formation. They are specialized fibroblasts that in addition to fibroblastic products, express bone sialoprotein and osteocalcin. Osteoblasts produce a matrix of osteoid, which is composed mainly of Type I collagen. Osteoblasts are also responsible for mineralization of this matrix. Zinc, copper and sodium are some of the minerals required in this process. Bone is a dynamic tissue that is constantly being reshaped by osteoblasts, which are in charge of production of matrix and mineral, and osteoclasts, which break down the tissue. The number of osteoblasts tends to decrease with age, affecting the balance of formation and resorption in the bone tissue, and potentially leading to osteoporosis.

Osteoblasts arise from osteoprogenitor cells located in the deeper layer of periosteum and the bone marrow. Osteoprogenitors are immature progenitor cells that express the master regulatory transcription factor Cbfa1/Runx2. Osteoprogenitors are induced to differentiate under the influence of growth factors, in particular the bone morphogenetic proteins (BMPs). Aside from BMPs, other growth factors including fibroblast growth factor (FGF), platelet-derived growth factor (PDGF) and transforming growth factor beta (TGF-β) may promote the division of osteoprogenitors and potentially increase osteogenesis. Once osteoprogenitors start to differentiate into osteoblasts, they begin to express a range of genetic markers including Osterix, Col1, BSP, M-CSF, ALP, osteocalcin, osteopontin, and osteonectin. Although the term osteoblast implies an immature cell type, osteoblasts are in fact the mature bone cells entirely responsible for generating bone tissue in animals and humans.

Cronstein, U.S. Pat. No. 7,795,427 describes the use of agents that block adenosine A₁ receptor antagonists to diminish osteoclast function and thereby prevent the development of osteoporosis. Cronstein, U.S. Pat. No. 8,183,225 (U.S. Ser. No. 12/291,510) describes the activation of adenosine A_{2A} receptors as inhibiting osteoclast formation and function, and use of adenosine A_{2A} receptor agonists to prevent wear particle-induced bone resorption. In all of these actions adenosine receptor blockade or activation was directed solely at preventing bone resorption. Interestingly, neither adenosine A₁ nor A_{2A} receptors may affect the formation or function of osteoblasts.

The prior art teaches use of adenosine receptor agonists and antagonists or dipyridamole in the regulation of osteoblast differentiation, proliferation and function. Dipyridamole is described in the prior art to increase adenosine to stimulate adenosine A_{2B} receptors to stimulate osteoblast production of bone matrix and inhibit IL-6 production or increase production of osteoprotegerin. (See, e.g., Kara et al., The FASEB Journal 2010; 24: 2325-2333; Kara et al., Arthritis and Rheumatism 2010; 62:534-541; Russell et al., Calcif Tissue Int 2007; 81:316-326; Evans et al., J Bone Miner Res 2006; 21: 228-236; Costa et al., Journal of Cellular Physiology 2011; 226: 1353-1366) Cronstein et al. teach using adenosine A₁ or A_{2A} receptor agonists or antagonists or dipyridamole for the treatment of bone defects following trauma or to promote spinal fusion in PCT/US2013/027097, filed Feb. 21, 2013, and U.S. Ser. No. 14/380,238, filed Aug. 21, 2014, the disclosures of which are incorporated herein by reference in its entirety.

### SUMMARY OF THE INVENTION

In a first aspect, the present invention provides a tissue repair device or scaffold having a porous bone ingrowth area containing interconnected struts surrounded by a microporous shell having therein or thereon a therapeutically effective amount of an adenosine receptor agonist or an adenosine receptor antagonist, or an agent that upregulates, increases the amount of or increases the biological activity of adenosine or an analog or derivative thereof. The therapeutically effective amount of an adenosine receptor agonist or an adenosine receptor antagonist, or an agent that upregulates, increases the amount of or increases the biological activity of adenosine or an analog or derivative thereof may be provided in a timed release or sustained release formulation, such as, for instance a collagen solution, such as a 1%, 2%, 3%, 4%, 5%, 10% or so collagen solution. The microporous shell may function to attach but limit soft tissue ingrowth. At the ends of the tissue repair device or scaffold, the shell may be extended as a guide flange to stabilize the tissue repair device or scaffold between ends of bone, across a bone defect, etc. or the tissue repair device or scaffold may be used to repair a defect of a flat bone. The center of the tissue repair device or scaffold may be empty and may serve as a potential marrow space. The porous ingrowth structure may be infiltrated with a soluble filler or carrier, such as, for example calcium sulfate. This soluble filler or carrier, such as, for example calcium sulfate, may be infiltrated with one or more of an antibiotic, a growth factor, a differentiation factor, a cytokine, a drug, or a combination of these agents. The tissue repair device or scaffold may fit between the cortical bone ends of long bone and conduct healing bone, which arises largely from the endosteal and periosteal surfaces or it may be used at or near a bone defect of, for instance, flat bone. The tissue repair device or scaffold may be stabilized using a modified bone plate or bone screws. The tissue repair device or scaffold may be produced by a three dimensional printing procedure and may be formed of, for instance, an osteoconductive ceramic.

The tissue repair device or scaffold may be a multiphasic, three-dimensionally printed, tissue repair device. The struts may be substantially cylindrical and they may be, for instance, from about 1-1,000, 10-900, 20-800, 30-700, 40-600, 50-500, 60-400, 100-350, 120-300, or about 200-275 μm diameter. In some embodiments, the struts may be about 20-940 μm diameter. In some embodiments the struts are within about 3×, 2× or 1.5× or substantially the same diameter as bone trabeculae. In some embodiments, the struts may be separated longitudinally by a space of up to 100, 200, 300, 400, 500, 600, 700, 800, 900 μm or more, or even 1.0 mm or more. Similarly, the tissue repair device or scaffold may be porous having mesopores that may be present in a size generally less than about 100, 75, 50, 30, 20, 10 or even less than about 5, 4, 3, 2, 1, or even 0.5, 0.4, 0.3, 0.2 or 0.1 μm diameter. The struts may be arranged in a substantially linear arrangement. The tissue repair device or scaffold may be substantially resorbable so that, for instance, after about 8, 10, 12, 16, 18, 20, 24 or so weeks presence in vivo, about 5, 10, 15, 20, 25, 30, 35, 40, 45, or 50% or more of the tissue repair device or scaffold may be resorbed. The tissue repair device or scaffold may be at least about 50%, 60%, 70%, 75%, 80%, 85%, 90%, 95% or even more porous. Similarly, the tissue repair device or scaffold may be efficient to encourage and provide bone growth such that after about 8 or 16 weeks presence in vivo, about 5, 10, 15, 20, 25, 30, 35, 40, 45, or 50% or more of the tissue repair device or scaffold may be replaced by bone. The tissue repair device or scaffold may promote or form cancellar or cortical bone, within the tissue repair device or scaffold or in the region or area of the tissue repair device or scaffold. The tissue repair device or scaffold may be used to remodel bone or to regionally control the density of bone.

The tissue repair device or scaffold may feature a gradient of mesopores formed by varying strut spacing in three dimensions (X, Y, and Z). Spacing in the X and Y dimensions may be accomplished using radial or V-shaped patterns with spacing from, for instance, 100-940 μm. Spacing in the Z dimension may be accomplished by stacking multiple layers of the radial struts. The porous ingrowth structure may be infiltrated with a soluble filler or carrier, such as, for example calcium sulfate. In some embodiments, the porous ingrowth structure may be infiltrated with a filler that attracts osteoclasts, such as, for example calcium phosphate mineral and type I collagen protein. In some instances, the printed tissue repair device or scaffold s may be micro/nanoporous on about a 0.1-1 μm pore size level. The pores then may in some instances be infiltrated with solubilized collagen.

The tissue repair device or scaffold may be effective for promoting bone growth and treating bone fracture, defect or deficiency across a distance of at least 5, 10, 11, 12, 13, 14, 15, 18, 20, 25, 30, 35, 40, 50, 60, 70, 80, 90 or 100 or more millimeters. Similarly, the tissue repair device or scaffold may be effective for promoting the growth of both cortical or cortical-like bone and trabecular or trabecular-like bone. The bone so grown may be in any suitable proportion, such as, for example 95%, 90%, 80%, 75%, 70%, 60%, 50%, 40%, 30%, 25%, 20%, 10% or so trabecular or trabecular-like bone, or just the opposite, i.e. 95%, 90%, 80%, 75%, 70%, 60%, 50%, 40%, 30%, 25%, 20%, 10% or so cortical or cortical-like bone. The tissue repair device or scaffold may be effective for reducing or shortening the normal repair time across a bone defect by 5, 10, 20, 25, 30, 40, 50, 75, 90% or more. In some instances, the bone defect may be repaired in about half, one third or one quarter of the normally required period of time. In many instances, the larger pore sizes are found near the outer portions of the scaffold and the smaller pore sizes are found near the inner portions of the scaffold. In some instances, the portion of the scaffold forming the inner half of the surface area may have a median pore diameter size or area that is 5, 10, 20, 25, 30, 40, 50, 75, 90% or more smaller than the median pore diameter size or area of the portion of the scaffold forming the outer half of the surface area. In some instances the pore sizes are arranged architecturally in any suitable or desirable configuration so as to customize the type of bone growth, for instance bone density, trabecular-like bone or cortical-like bone, desired. Similarly, in some instances, the tissue repair device or scaffold is formed and shaped to customize the shape of tissue or bone repair desired to optimally span a defect. Further, in some instances, a portion of the tissue repair device or scaffold may be substantially hollow, for instance, 10, 20, 25, 30, 40, 50, 75, 90% or more of the interior portion of the tissue repair device or scaffold may be substantially hollow.

The adenosine receptor of the present invention may be any one of A₁, A_{2A}, A_{2B} or A₃. In a more particular embodiment, the adenosine receptor is an A_{2A} receptor, and the agonist is an adenosine receptor A_{2A} agonist. In another more particular embodiment, the adenosine receptor is an A_{2B} receptor, and the agonist is an adenosine receptor A_{2B} agonist. In yet another embodiment, the adenosine receptor agonist affects more than one adenosine receptor. In a more particular embodiment, the adenosine receptor is an A₁ receptor, and the antagonist is an adenosine receptor A₁ antagonist. In yet another embodiment, the adenosine receptor antagonist affects more than one adenosine receptor.

In another particular embodiment, the adenosine receptor agonist is a selective adenosine receptor agonist. In still other particular embodiments, the adenosine receptor agonist is a non-selective adenosine receptor agonist. In another particular embodiment, the adenosine receptor antagonist is a selective adenosine receptor agonist. In still other particular embodiments, the adenosine receptor antagonist is a non-selective adenosine receptor antagonist.

In a more particular embodiment, the agent that agonizes an adenosine receptor is an adenosine A_{2A} receptor agonist or an adenosine A_{2B} receptor agonist. The adenosine receptor agonist may be, for instance, a small organic molecule, a protein or peptide, a nucleic acid or an antibody. Similarly, in a more particular embodiment, the agent that antagonizes an adenosine receptor is an adenosine A₁ receptor antagonist. The adenosine receptor antagonist may be, for instance, a small organic molecule, a protein or peptide, a nucleic acid or an antibody.

In yet another more particular embodiment, the adenosine receptor agonist is capable of substantially stimulating the endogenous activity of the adenosine receptor substantially the same as though the adenosine receptor had encountered its natural, endogenous ligand.

In yet another particular embodiment, the adenosine receptor agonist or antagonist is an adenosine A_{2A} receptor agonist or an adenosine A₁ receptor antagonist.

In some instances, the bone regeneration or differentiation or activation of osteoblasts may be increased by 10%, 20%, 30%, 40%, 50%, 60%, 70%, 80%, 90%, 100%, or by two fold, three fold, four fold, five fold, ten fold or more relative to normal. Likewise, in some instances, the speed of bone regeneration or number of differentiated or stimulated osteoblasts may be increased by 10%, 20%, 30%, 40%, 50%, 60%, 70%, 80%, 90%, 100%, or by two fold, three fold, four fold, five fold, ten fold or more relative to normal.

In one particular embodiment, an effective amount of an adenosine receptor agonist or antagonist or an agent that upregulates, increases the amount of or increases the biological activity of adenosine or an analog or derivative thereof, may be used in combination with one or more drugs useful in inhibiting bone resorption or inhibiting differentiation or stimulation of osteoclasts or in stimulating bone regeneration or growth or stimulating or promoting differentiation of osteoblasts or a combination of any of these agents.

Adenosine A_{2A} receptor agonists are well known in the art. Many are disclosed in, for instance, U.S. Pat. Nos. 7,226,913 and 6,326,359 and in United States Patent Publication Nos. 20070225247, 20060100169, 20060034941, 20050261236, 20050182018, 20050171050, 20050020915 and 20040064039, the disclosures of which are herein incorporated by reference in their entireties. In another more particular embodiment, the adenosine A_{2A} receptor agonist is selected from the group consisting of CGS 21680, MRE-0094, IB-MECA and R-PIA, binodenoson, ATL146, for instance. Adenosine A_{2B} receptor agonists are also known in the art. Many are disclosed in, for instance, United States Patent Publication Nos. 20070225335 and 20070240433.

Likewise, adenosine A₁ receptor antagonists are well known in the art and include, for instance, DPCPX. Exemplary A₁ receptor antagonists include those disclosed by Cronstein, U.S. Pat. No. 7,795,427 such as DPCPX (8-Cyclopentyl-1,3-dipropylxanthine), N-0861 (N-6-endonorboman-2-yl-9-methyladenine), N-0840 (N-6-cyclopentyl-9-methyladenine), CVT-124, WRC-0342 ([N⁶-(5′-endohydroxy)-endonorbornan-2-yl-9-methyladenine]), CGS-15943, XAC (xanthine carboxylic acid congener), WRC-0571 ([C⁸—(N-methylisopropyl)-amino-N⁶(5′-endohydroxy)-endonorbornan- -2-yl-9-methyladenine],), KW-3902 (8-(noradamantan-3-yl)-1,3-dipropylxanthine), ENX (1,3-Dipropyl-8-[2-(5,6-epoxy)norbornyl]xanthine), KFM 19 (BIIP20, (S)-3,7-dihydro-8-(3-oxocyclopentyl)-1,3-dipropyl-1H-purine-2,6-dione), FK453 ((R)-1-[(E)-3-(2-phenylpyrazolo[1,5-a]pyridin-3-yl) acryloyl]-2-piperidine ethanol), FK352 ((R)-1-[(E)-3-(2-phenylpyrazolo[1,5-a]pyridin-3-yl) acryloyl]-piperidin-2-yl acetic acid), FK838 (6-oxo-3-(2-phenylpyrazolo[1,5-a]pyridin-3-yl)-1(6H)-pyridazinebutyric acid), FR166124 and its analogues, 8-cyclopentyltheophylline, BG9719 and BG9928.

The adenosine receptor agonist or antagonist may be present in combination with one or more other compounds or agents for inhibiting bone resorption or osteoclast differentiation or for stimulating or increasing bone regeneration or bone growth or stimulating osteoblast differentiation. Such other compounds may be, for instance, anti-inflammatory compounds, bisphosphonates or growth factors. The adenosine receptor agonist may be administered with a second adenosine receptor agonist or with a less selective adenosine receptor agonist. (i.e. one that binds other adenosine receptors in addition to A_{2A} or A_{2B} for example A_{2B}, A₁ or A₃). The adenosine receptor agonist or antagonist or an agent that upregulates, increases the amount of or increases the biological activity of adenosine or an analog or derivative thereof, may be administered or provided in a matrix, including a matrix designed to provide timed or sustained release, such as, for example a calcium sulfate matrix, a calcium phosphate matrix or bovine collagen.

In one embodiment, the adenosine receptor agonist may be selective for the receptor, or it may be a non-selective adenosine receptor agonist, which may stimulate or mimic natural ligands of one or more of the following receptors: A₁, A_{2A}, A_{2B} or A₃. In a preferred embodiment, the adenosine receptor agonist is an adenosine A_{2A} receptor agonist.

In another more particular embodiment, the agent that increases endogenous adenosine levels may be an agent that, for instance, diminishes platelet function or induces coronary vasodilation, such as, for instance, dipyridamole or ticagrelor.

In a second aspect, the present invention provides a method for promoting bone growth or treating bone fracture, defect or deficiency by providing a tissue repair device or scaffold having a porous bone ingrowth area containing interconnected struts surrounded by a microporous shell having therein or thereon a therapeutically effective amount of an adenosine receptor agonist or an adenosine receptor antagonist, or an agent that upregulates, increases the amount of or increases the biological activity of adenosine or an analog or derivative thereof. The therapeutically effective amount of an adenosine receptor agonist or an adenosine receptor antagonist, or an agent that upregulates, increases the amount of or increases the biological activity of adenosine or an analog or derivative thereof may be provided in a timed release or sustained release formulation, such as, for instance a collagen solution, such as a 1%, 2%, 3%, 4%, 5%, 10% or so collagen solution. The promoting bone growth or treating bone fracture, defect or deficiency may feature controlling or affecting the density of bone or may feature remodeling bone, for instance, cancellar or cortical bone. In most instances the tissue repair device or scaffold is provided in vivo to a region featuring a bone deficiency, fracture or void. The microporous shell may function to attach but limit soft tissue ingrowth. At the ends of the tissue repair device or scaffold, the shell may be extended as a guide flange to stabilize the tissue repair device or scaffold between ends of bone. The center of the tissue repair device or scaffold may be empty and may serve as a potential marrow space. The porous ingrowth structure may be infiltrated with a soluble filler or carrier, such as, for example calcium sulfate. This soluble filler or carrier, such as, for example calcium sulfate, may be infiltrated with one or more of an antibiotic, a growth factor, a differentiation factors, a cytokine, a drug, or a combination of these agents. The tissue repair device or scaffold may fit between the cortical bone ends of long bone and conduct healing bone, which arises largely from the endosteal and periosteal surfaces. The tissue repair device or scaffold may be stabilized using a modified bone plate or bone screws. The tissue repair device or scaffold may be produced by a three dimensional printing procedure and may be formed of, for instance, an osteoconductive ceramic.

The tissue repair device or scaffold may be a multiphasic, three-dimensionally printed, tissue repair device. The struts may be substantially cylindrical and they may be, for instance, from about 1-1,000, 10-900, 20-800, 30-700, 40-600, 50-500, 60-400, 100-350, 120-300, or about 200-275 μm diameter. In some embodiments the struts are about 20-940 μm diameter. In some embodiments the struts are within about 3×, 2× or 1.5× or substantially the same diameter as bone trabeculae. In some embodiments, the struts may be separated longitudinally by a space of up to 100, 200, 300, 400, 500, 600, 700, 800, 900 μm or more, or even 1.0 mm or more. Similarly, the tissue repair device or scaffold may be porous having mesopores that may be present in a size generally less than about 100, 75, 50, 30, 20, 10 or even less than about 5, 4, 3, 2, 1, or even 0.5, 0.4, 0.3, 0.2 or 0.1 μm diameter. The struts may be arranged in a substantially linear arrangement. The tissue repair device or scaffold may be substantially resorbable so that, for instance, after about 8, 10, 12, 16, 18, 20, 24 or so weeks presence in vivo, about 5, 10, 15, 20, 25, 30, 35, 40, 45, or 50% or more of the tissue repair device or scaffold may be resorbed. The tissue repair device or scaffold may be at least about 50%, 60%, 70%, 75%, 80%, 85%, 90%, 95% or even more porous. Similarly, the tissue repair device or scaffold may be efficient to encourage and provide bone growth such that after about 8 or 16 weeks presence in vivo, about 5, 10, 15, 20, 25, 30, 35, 40, 45, or 50% or more of the tissue repair device or scaffold may be replaced by bone.

The tissue repair device or scaffold may feature a gradient of mesopores formed by varying strut spacing in three dimensions (X, Y, and Z). Spacing in the X and Y dimensions may be accomplished using radial or V-shaped patterns with spacing from, for instance, 100-940 μm. Spacing in the Z dimension may be accomplished by stacking multiple layers of the radial struts. The porous ingrowth structure may be infiltrated with a soluble filler or carrier, such as, for example calcium sulfate. In some embodiments, the porous ingrowth structure may be infiltrated with a filler that attracts osteoclasts, such as, for example calcium phosphate mineral and type I collagen protein. In some instances, the printed tissue repair device or scaffolds may be micro/nanoporous on about a 0.1-1 μm pore size level. The pores then may in some instances be infiltrated with solubilized collagen.

The tissue repair device or scaffold may be effective for promoting bone growth and treating bone fracture, defect or deficiency across a distance of at least 5, 10, 11, 12, 13, 14, 15, 18, 20, 25, 30, 35, 40, 50, 60, 70, 80, 90 or 100 or more millimeters. Similarly, the tissue repair device or scaffold may be effective for promoting the growth of both cortical or cortical-like bone and trabecular or trabecular-like bone. The bone so grown may be in any suitable proportion, such as, for example 95%, 90%, 80%, 75%, 70%, 60%, 50%, 40%, 30%, 25%, 20%, 10% or so trabecular or trabecular-like bone, or just the opposite, i.e. 95%, 90%, 80%, 75%, 70%, 60%, 50%, 40%, 30%, 25%, 20%, 10% or so cortical or cortical-like bone. The tissue repair device or scaffold may be effective for reducing or shortening the normal repair time across a bone defect by 5, 10, 20, 25, 30, 40, 50, 75, 90% or more. In some instances, the bone defect may be repaired in about half, one third or one quarter of the normally required period of time. In many instances, the larger pore sizes are found near the outer portions of the scaffold and the smaller pore sizes are found near the inner portions of the scaffold. In some instances, the portion of the scaffold forming the inner half of the surface area may have a median pore diameter size or area that is 5, 10, 20, 25, 30, 40, 50, 75, 90% or more smaller than the median pore diameter size or area of the portion of the scaffold forming the outer half of the surface area. In some instances the pore sizes are arranged architecturally in any suitable or desirable configuration so as to customize the type of bone growth, for instance bone density, trabecular-like bone or cortical-like bone, desired. Similarly, in some instances, the tissue repair device or scaffold is formed and shaped to customize the shape of tissue or bone repair desired to optimally span a defect.

The adenosine receptor of the present invention may be any one of A₁, A_{2A}, A_{2B} or A₃. In a more particular embodiment, the adenosine receptor is an A_{2A} receptor, and the agonist is an adenosine receptor A_{2A} agonist. In another more particular embodiment, the adenosine receptor is an A_{2B} receptor, and the agonist is an adenosine receptor A_{2B} agonist. In yet another embodiment, the adenosine receptor agonist affects more than one adenosine receptor. In a more particular embodiment, the adenosine receptor is an A₁ receptor, and the antagonist is an adenosine receptor A₁ antagonist. In yet another embodiment, the adenosine receptor antagonist affects more than one adenosine receptor.

In another particular embodiment, the adenosine receptor agonist is a selective adenosine receptor agonist. In still other particular embodiments, the adenosine receptor agonist is a non-selective adenosine receptor agonist. In another particular embodiment, the adenosine receptor antagonist is a selective adenosine receptor agonist. In still other particular embodiments, the adenosine receptor antagonist is a non-selective adenosine receptor antagonist.

In a more particular embodiment, the agent that agonizes an adenosine receptor is an adenosine A_{2A} receptor agonist or an adenosine A_{2B} receptor agonist. The adenosine receptor agonist may be, for instance, a small organic molecule, a protein or peptide, a nucleic acid or an antibody. Similarly, in a more particular embodiment, the agent that antagonizes an adenosine receptor is an adenosine A₁ receptor antagonist. The adenosine receptor antagonist may be, for instance, a small organic molecule, a protein or peptide, a nucleic acid or an antibody.

In yet another more particular embodiment, the adenosine receptor agonist is capable of substantially stimulating the endogenous activity of the adenosine receptor substantially the same as though the adenosine receptor had encountered its natural, endogenous ligand.

In yet another particular embodiment, the adenosine receptor agonist or antagonist is an adenosine A_{2A} receptor agonist or an adenosine A₁ receptor antagonist.

In some instances, the bone regeneration or differentiation or activation of osteoblasts may be increased by 10%, 20%, 30%, 40%, 50%, 60%, 70%, 80%, 90%, 100%, or by two fold, three fold, four fold, five fold, ten fold or more relative to normal. Likewise, in some instances, the speed of bone regeneration or number of differentiated or stimulated osteoblasts may be increased by 10%, 20%, 30%, 40%, 50%, 60%, 70%, 80%, 90%, 100%, or by two fold, three fold, four fold, five fold, ten fold or more relative to normal.

In one particular embodiment, an effective amount of an adenosine receptor agonist or antagonist may be used in combination with one or more drugs useful in inhibiting bone resorption or inhibiting differentiation or stimulation of osteoclasts or in stimulating bone regeneration or growth or stimulating or promoting differentiation of osteoblasts or a combination of any of these agents.

Adenosine A_{2A} receptor agonists are well known in the art. Many are disclosed in, for instance, U.S. Pat. Nos. 7,226,913 and 6,326,359 and in United States Patent Publication Nos. 20070225247, 20060100169, 20060034941, 20050261236, 20050182018, 20050171050, 20050020915 and 20040064039, the disclosures of which are herein incorporated by reference in their entireties. In another more particular embodiment, the adenosine A_{2A} receptor agonist is selected from the group consisting of CGS 21680, MRE-0094, IB-MECA and R-PIA, binodenoson, ATL146, for instance. Adenosine A_{2B} receptor agonists are also known in the art. Many are disclosed in, for instance, United States Patent Publication Nos. 20070225335 and 20070240433.

Likewise, adenosine A₁ receptor antagonists are well known in the art and include, for instance, DPCPX. Exemplary A₁ receptor antagonists include those disclosed by Cronstein, U.S. Pat. No. 7,795,427 such as DPCPX (8-Cyclopentyl-1,3-dipropylxanthine), N-0861 (N-6-endonorboman-2-yl-9-methyladenine), N-0840 (N-6-cyclopentyl-9-methyladenine), CVT-124, WRC-0342 ([N⁶-(5′-endohydroxy)-endonorbornan-2-yl-9-methyladenine]), CGS-15943, XAC (xanthine carboxylic acid congener), WRC-0571 ([C⁸—(N-methylisopropyl)-amino-N⁶(5′-endohydroxy)-endonorbornan- -2-yl-9-methyladenine],), KW-3902 (8-(noradamantan-3-yl)-1,3-dipropylxanthine), ENX (1,3-Dipropyl-8-[2-(5,6-epoxy)norbornyl]xanthine), KFM 19 (BIIP20, (S)-3,7-dihydro-8-(3-oxocyclopentyl)-1,3-dipropyl-1H-purine-2,6-dione), FK453 ((R)-1-[(E)-3-(2-phenylpyrazolo[1,5-a]pyridin-3-yl) acryloyl]-2-piperidine ethanol), FK352 ((R)-1-[(E)-3-(2-phenylpyrazolo[1,5-a]pyridin-3-yl) acryloyl]-piperidin-2-yl acetic acid), FK838 (6-oxo-3-(2-phenylpyrazolo[1,5-a]pyridin-3-yl)-1(6H)-pyridazinebutyric acid), FR166124 and its analogues, 8-cyclopentyltheophylline, BG9719 and BG9928.

The adenosine receptor agonist or antagonist may be present in combination with one or more other compounds or agents for inhibiting bone resorption or osteoclast differentiation or for stimulating or increasing bone regeneration or bone growth or stimulating osteoblast differentiation. Such other compounds may be, for instance, anti-inflammatory compounds, bisphosphonates or growth factors. The adenosine receptor agonist may be administered with a second adenosine receptor agonist or with a less selective adenosine receptor agonist. (i.e. one that binds other adenosine receptors in addition to A_{2A} or A_{2B} for example A_{2B}, A₁ or A₃). The adenosine receptor agonist or antagonist may be administered or provided in a matrix, including a matrix designed for timed or sustained release, such as, for example a calcium sulfate matrix, a calcium phosphate matrix or bovine collagen.

In one embodiment, the adenosine receptor agonist may be selective for the receptor, or it may be a non-selective adenosine receptor agonist, which may stimulate or mimic natural ligands of one or more of the following receptors: A₁, A_{2A}, A_{2B} or A₃. In a preferred embodiment, the adenosine receptor agonist is an adenosine A_{2A} receptor agonist.

In another more particular embodiment, the agent that increases endogenous adenosine levels may be an agent that, for instance, diminishes platelet function or induces coronary vasodilation, such as, for instance, dipyridamole or ticagrelor.

In a third aspect, the present invention provides a method for producing a tissue repair device or scaffold useful for promoting bone growth or treating bone fracture, defect or deficiency having a porous bone ingrowth area containing interconnected struts surrounded by a microporous shell having therein or thereon a therapeutically effective amount of an adenosine receptor agonist or an adenosine receptor antagonist, or an agent that upregulates, increases the amount of or increases the biological activity of adenosine or an analog or derivative thereof. The method features (a) providing a microporous shell that may function to attach but limit soft tissue ingrowth, (b) infiltrating a porous ingrowth structure with a soluble filler or carrier, (c) applying or providing an adenosine receptor agonist or an adenosine receptor antagonist, or an agent that upregulates, increases the amount of or increases the biological activity of adenosine or an analog or derivative thereof, and optionally (d) infiltrating the porous ingrowth structure with one or more of an antibiotic, a growth factor, a differentiation factor, a cytokine, a drug, or a combination of these agents. The soluble filler or carrier may be a filler that attracts osteoclasts, such as, for example calcium phosphate mineral and type I collagen protein. Tissue repair device or scaffold useful for promoting bone growth or treating bone fracture, defect or deficiency having a porous bone ingrowth area containing interconnected struts surrounded by a microporous shell may have the features described herein.

The adenosine receptor of the present invention may be any one of A₁, A_{2A}, A_{2B} or A₃. In a more particular embodiment, the adenosine receptor is an A_{2A} receptor, and the agonist is an adenosine receptor A_{2A} agonist. In another more particular embodiment, the adenosine receptor is an A_{2B} receptor, and the agonist is an adenosine receptor A_{2B} agonist. In yet another embodiment, the adenosine receptor agonist affects more than one adenosine receptor. In a more particular embodiment, the adenosine receptor is an A₁ receptor, and the antagonist is an adenosine receptor A₁ antagonist. In yet another embodiment, the adenosine receptor antagonist affects more than one adenosine receptor.

In another particular embodiment, the adenosine receptor agonist is a selective adenosine receptor agonist. In still other particular embodiments, the adenosine receptor agonist is a non-selective adenosine receptor agonist. In another particular embodiment, the adenosine receptor antagonist is a selective adenosine receptor agonist. In still other particular embodiments, the adenosine receptor antagonist is a non-selective adenosine receptor antagonist.

In a more particular embodiment, the agent that agonizes an adenosine receptor is an adenosine A_{2A} receptor agonist or an adenosine A_{2B} receptor agonist. The adenosine receptor agonist may be, for instance, a small organic molecule, a protein or peptide, a nucleic acid or an antibody. Similarly, in a more particular embodiment, the agent that antagonizes an adenosine receptor is an adenosine A₁ receptor antagonist. The adenosine receptor antagonist may be, for instance, a small organic molecule, a protein or peptide, a nucleic acid or an antibody.

In yet another more particular embodiment, the adenosine receptor agonist is capable of substantially stimulating the endogenous activity of the adenosine receptor substantially the same as though the adenosine receptor had encountered its natural, endogenous ligand.

In yet another particular embodiment, the adenosine receptor agonist or antagonist is an adenosine A_{2A} receptor agonist or an adenosine A₁ receptor antagonist.

In some instances, the bone regeneration or differentiation or activation of osteoblasts may be increased by 10%, 20%, 30%, 40%, 50%, 60%, 70%, 80%, 90%, 100%, or by two fold, three fold, four fold, five fold, ten fold or more relative to normal. Likewise, in some instances, the speed of bone regeneration or number of differentiated or stimulated osteoblasts may be increased by 10%, 20%, 30%, 40%, 50%, 60%, 70%, 80%, 90%, 100%, or by two fold, three fold, four fold, five fold, ten fold or more relative to normal.

In one particular embodiment, an effective amount of an adenosine receptor agonist or antagonist may be used in combination with one or more drugs useful in inhibiting bone resorption or inhibiting differentiation or stimulation of osteoclasts or in stimulating bone regeneration or growth or stimulating or promoting differentiation of osteoblasts or a combination of any of these agents.

Adenosine A_{2A} receptor agonists are well known in the art. Many are disclosed in, for instance, U.S. Pat. Nos. 7,226,913 and 6,326,359 and in United States Patent Publication Nos. 20070225247, 20060100169, 20060034941, 20050261236, 20050182018, 20050171050, 20050020915 and 20040064039, the disclosures of which are herein incorporated by reference in their entireties. In another more particular embodiment, the adenosine A_{2A} receptor agonist is selected from the group consisting of CGS 21680, MRE-0094, IB-MECA and R-PIA, binodenoson, ATL146, for instance. Adenosine A_{2B} receptor agonists are also known in the art. Many are disclosed in, for instance, United States Patent Publication Nos. 20070225335 and 20070240433.

Likewise, adenosine A₁ receptor antagonists are well known in the art and include, for instance, DPCPX. Exemplary A₁ receptor antagonists include those disclosed by Cronstein, U.S. Pat. No. 7,795,427 such as DPCPX (8-Cyclopentyl-1,3-dipropylxanthine), N-0861 (N-6-endonorboman-2-yl-9-methyladenine), N-0840 (N-6-cyclopentyl-9-methyladenine), CVT-124, WRC-0342 ([N⁶-(5′-endohydroxy)-endonorbornan-2-yl-9-methyladenine]), CGS-15943, XAC (xanthine carboxylic acid congener), WRC-0571 ([C⁸—(N-methylisopropyl)-amino-N⁶(5′-endohydroxy)-endonorbornan- -2-yl-9-methyladenine],), KW-3902 (8-(noradamantan-3-yl)-1,3-dipropylxanthine), ENX (1,3-Dipropyl-8-[2-(5,6-epoxy)norbornyl]xanthine), KFM 19 (BIIP20, (S)-3,7-dihydro-8-(3-oxocyclopentyl)-1,3-dipropyl-1H-purine-2,6-dione), FK453 ((R)-1-[(E)-3-(2-phenylpyrazolo[1,5-a]pyridin-3-yl) acryloyl]-2-piperidine ethanol), FK352 ((R)-1-[(E)-3-(2-phenylpyrazolo[1,5-a]pyridin-3-yl) acryloyl]-piperidin-2-yl acetic acid), FK838 (6-oxo-3-(2-phenylpyrazolo[1,5-a]pyridin-3-yl)-1(6H)-pyridazinebutyric acid), FR166124 and its analogues, 8-cyclopentyltheophylline, BG9719 and BG9928.

The adenosine receptor agonist or antagonist may be present in combination with one or more other compounds or agents for inhibiting bone resorption or osteoclast differentiation or for stimulating or increasing bone regeneration or bone growth or stimulating osteoblast differentiation. Such other compounds may be, for instance, anti-inflammatory compounds, bisphosphonates or growth factors. The adenosine receptor agonist may be administered with a second adenosine receptor agonist or with a less selective adenosine receptor agonist. (i.e. one that binds other adenosine receptors in addition to A_{2A} or A_{2B} for example A_{2B}, A₁ or A₃). The adenosine receptor agonist or antagonist may be administered or provided in a matrix such as, for example a calcium sulfate matrix, a calcium phosphate matrix or bovine collagen. Such a matrix may be directly applied to bone defects to promote bone formation.

In one embodiment, the adenosine receptor agonist may be selective for the receptor, or it may be a non-selective adenosine receptor agonist, which may stimulate or mimic natural ligands of one or more of the following receptors: A₁, A_{2A}, A_{2B} or A₃. In a preferred embodiment, the adenosine receptor agonist is an adenosine A_{2A} receptor agonist.

In another more particular embodiment, the agent that increases endogenous adenosine levels may be an agent that, for instance, diminishes platelet function or induces coronary vasodilation, such as, for instance, dipyridamole or ticagrelor.

### BRIEF DESCRIPTION OF THE FIGURES

FIG. 1 is a schematic drawing of a tissue repair device or scaffold design that may be used to regenerate a long bone defect, showing its placement and fixation in the defect. The scaffold has a porous bone ingrowth area (A) containing interconnected 250 μm cylindrical struts surrounded by a microporous shell (B) to attach but limit soft tissue ingrowth. At the ends of the scaffold, the shell may be extended as a guide flange (C) to stabilize the construct between the bone ends. The center of the scaffold (D) may be left empty as a potential marrow space. The porous ingrowth structure (outlined with dashed line in upper left drawing) may be infiltrated with a soluble filler/carrier (such as calcium sulfate as an example) that may be infiltrated with one or more of an antibiotic, growth factors, differentiation factors, cytokines, drugs, or a combination of these agents. The microporous shell or struts may contain thereon an adenosine receptor agonist or an adenosine receptor antagonist, or an agent that upregulates, increases the amount of or increases the biological activity of adenosine or an analog or derivative thereof. The scaffold may fit between the cortical bone ends (E) of the long bone and conduct healing bone, which arises largely from the endosteal and periosteal surfaces (F). The construct may be stabilized using a modified bone plate (G) and bone screws (H).

FIG. 2 depicts a direct write (DW) printing apparatus based on the extrusion/deposition of colloidal inks as continuous filaments. DW requires minimal processing aids (i.e., polymers) in the ink for self-supporting filament/struts that will enable printing of the lattice structures required for bone scaffolds. The scaffolds are printed by ink extrusion on the XY plane, “writing” the bottom layer, then moving up in Z height to write additional layers until a three dimensional structure is formed. Post-processing of the printed green bodies requires binder burnout and sintering in a high temperature furnace. The resulting scaffolds are of high resolution and very reproducible.

FIG. 3 depicts one previous scaffold design for calvaria defects having an 11 mm disk with quadrants having different lattice spacings ranging from 250 μm to 400 μm. After 8 and 16 weeks in vivo the smaller-pore regions produced a different pattern of bone growth and scaffold resorption than the larger-pore regions

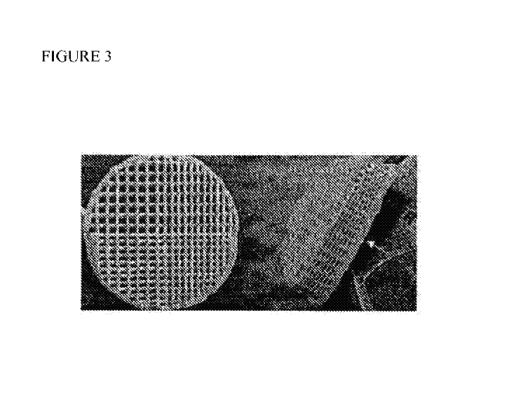

FIG. 4 depicts two scaffold architectures, (A) small-pore (SP) and (B) large-pore (LP), designed to increase the diversity of pore geometry. Both scaffolds contained a solid cap of layered parallel struts on one surface, which biologically served as a barrier to block soft tissue ingrowth from the scalp, but structurally served as a base for the printing of the scaffold lattice in the Z direction. The scaffold design built upon this base differed between the SP and LP scaffolds, but in general, consisted of a layers of nested concentric circles (CC) alternating with one or more radial (R) layers. Variation of porosity in the Z direction arose from use of 1, 2, or 3 stacks of radial layers, and porosity in the X and Y direction came from the spacing between radial struts in the same layer.

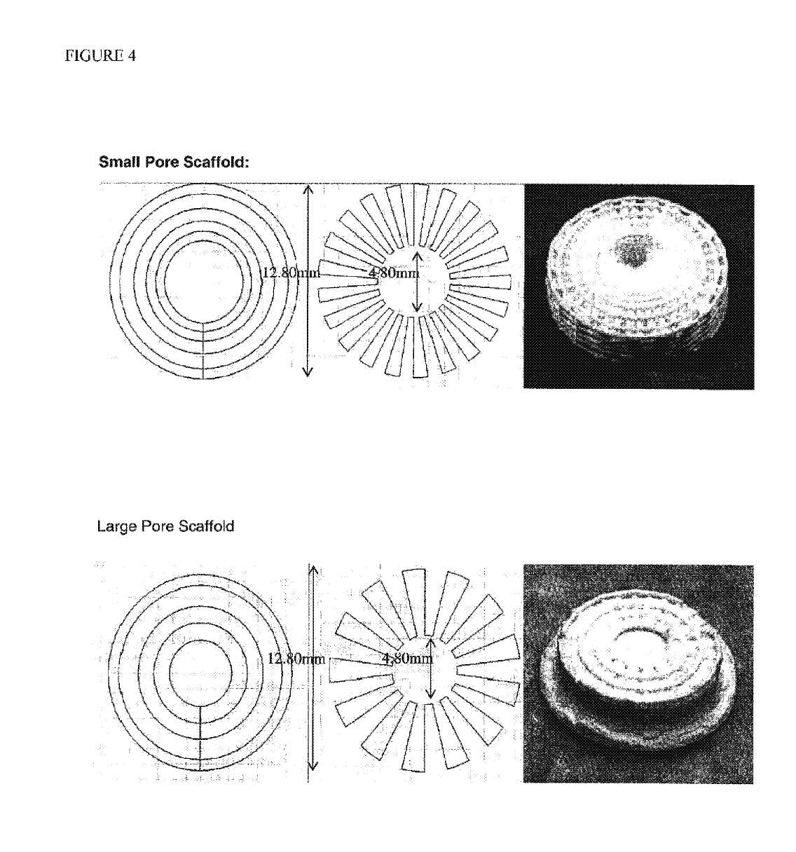

FIG. 5 provides a diagram of a unique mesopore volume formed. A ring of such volumes form the space between CC and R layers in the scaffolds described.

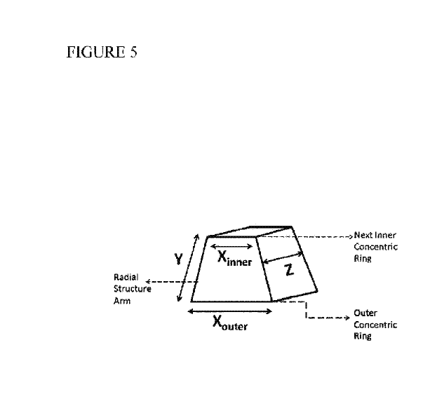

FIG. 6 shows (left) a horizontal slice from SP scaffold after 8 weeks through 1 Z height mesopores, with pores formed by concentric circle (CC) and radial (R) struts. All pores but the largest on the outside were evaluated with microCT. As R struts narrowed, bone began to attach to struts. Bone appears discontinuous because it grew upward from between CC rings, as shown in image on (upper-right), a vertical slice of 1 Z and 2 Z height mesopores at outer ring from same scaffold. (lower-right), horizontal slice of LP scaffold after 16 weeks. Note the significant formation osteoid (green) where resorbed struts are being replaced with new bone.

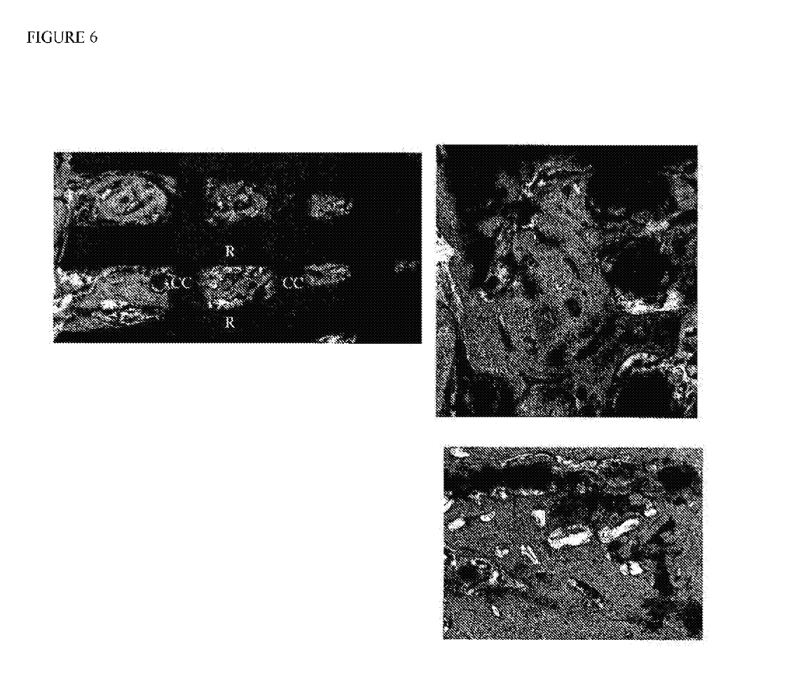

FIG. 7 provides the observed 1Z height mesopore percentages from a small pore scaffold having three ring sizes, large, middle and small, after 0, 8 and 16 weeks.

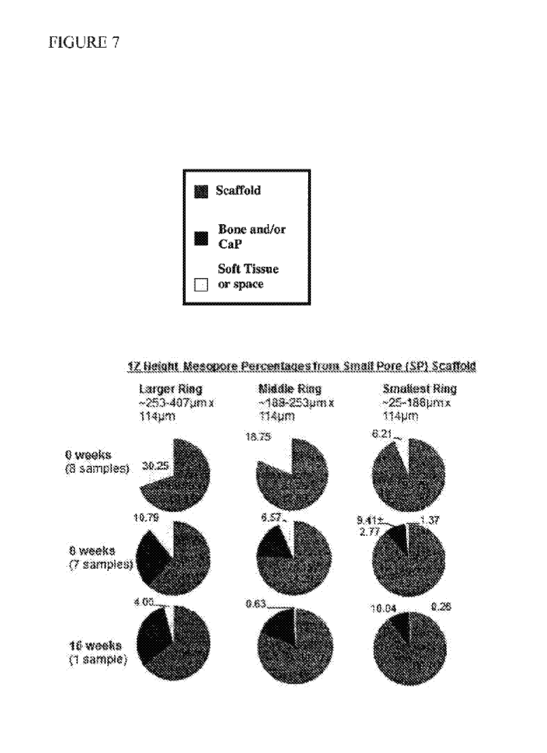

FIG. 8 provides a vertical slice through center and horizontal slices through middle of 3 Z mesopores in LP scaffold after 8 weeks.

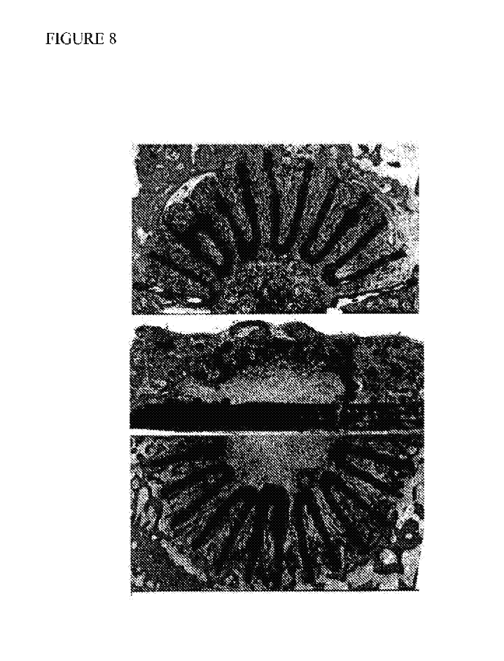

FIG. 9 provides the observed 2Z height mesopore percentages from a small pore scaffold having three ring sizes, large, middle and small, after 0, 8 and 16 weeks.

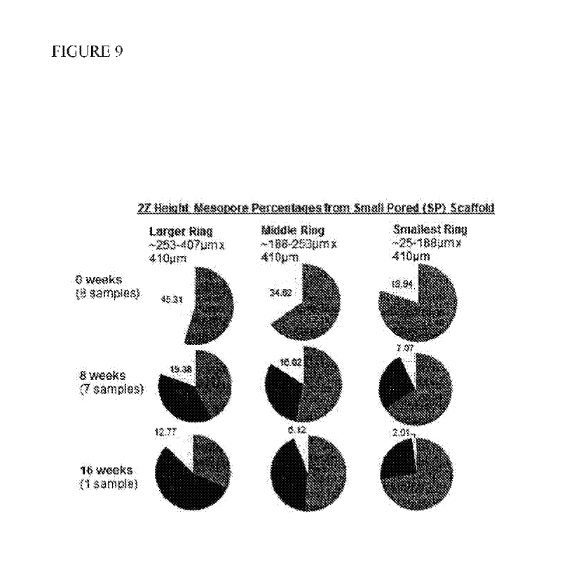

FIG. 10 provides the observed 3Z height mesopore percentages from a small pore scaffold having three ring sizes, large, middle and small, after 0, 8 and 16 weeks.

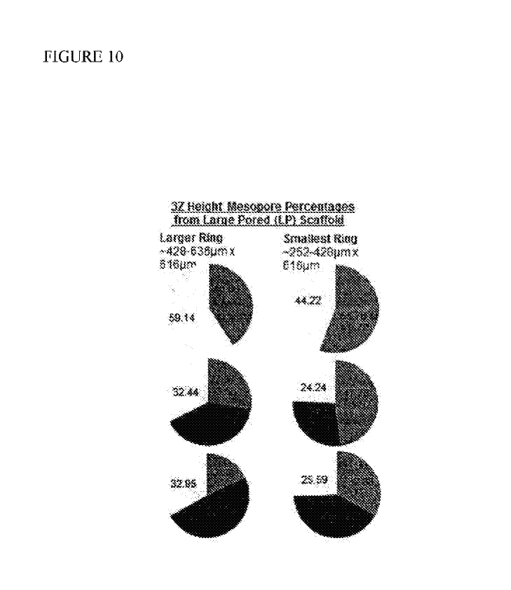

FIG. 11 depicts A) a large pore scaffold; B) a small pore scaffold, before and after removal of outer ring; and C) an enlarged diagram of an outer ring large mesopore. The rectangles correspond to the 3 layers of radial struts between concentric circles and the arrows designate the 4 open walls of the mesopore.

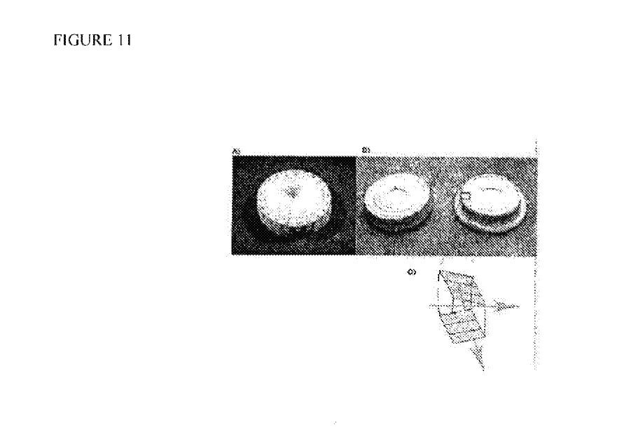

FIG. 12 provides a microCT scan of a large pore scaffold after 16 weeks. The scaffold is seen digitally sectioned both vertically through the center and horizontally between superficial and deep mesopores. The scaffold and cap appear darker, and the surrounding hard tissue appears in lighter shade.

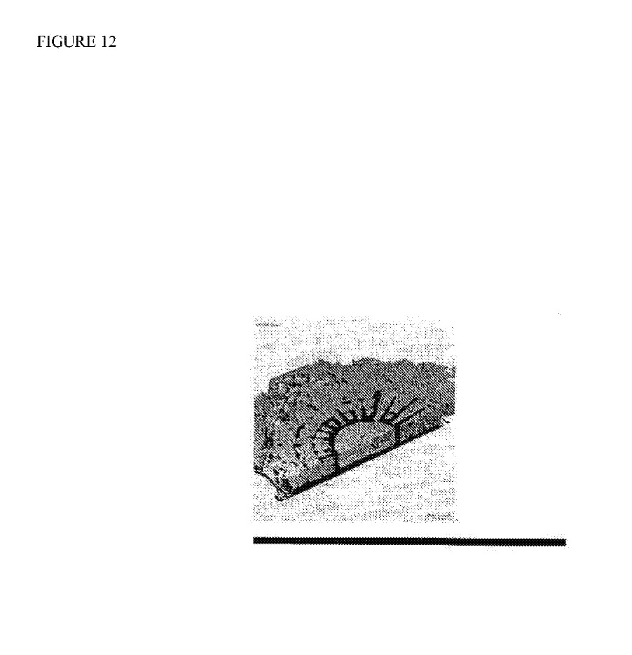

FIG. 13 shows horizontal slices from a scaffold through mesopores.

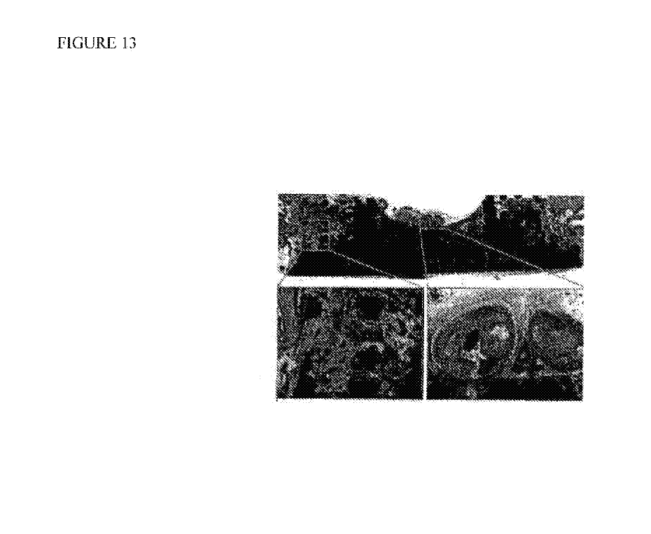

FIG. 14 provides a vertical slice through the center and horizontal slices through middle of 3 Z mesopores in a scaffold.

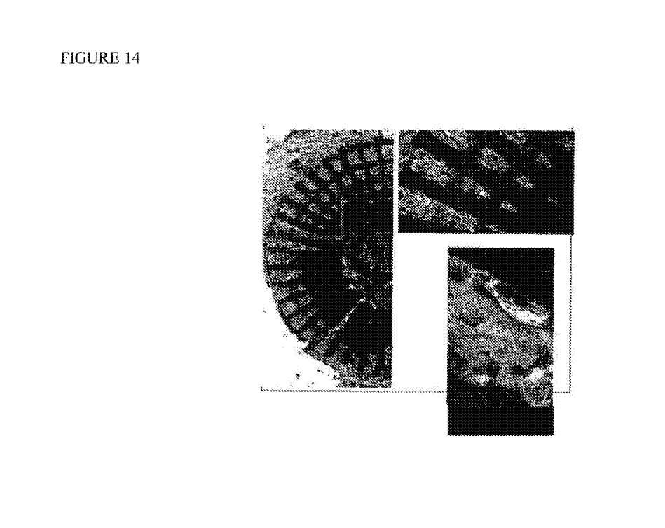

FIG. 15 depicts a scaffold having a four quadrant mesopore design having mesopores of differing sizes in distinct quadrants.

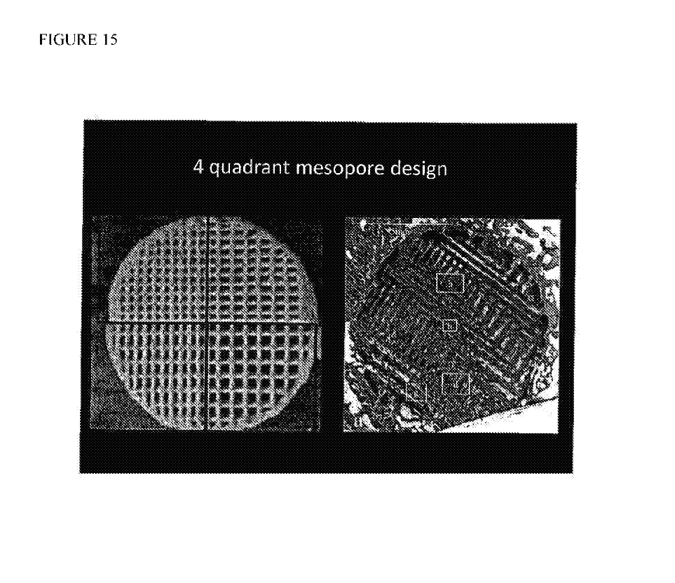

FIG. 16 depicts two scaffold architectures, (A) small-pore (SP) and (B) large-pore (LP), designed to increase the diversity of pore geometry. The small-pore design has pore dimensions of from 0-410 μm, and the large-pore design has pore dimensions of from 250-940 μm.

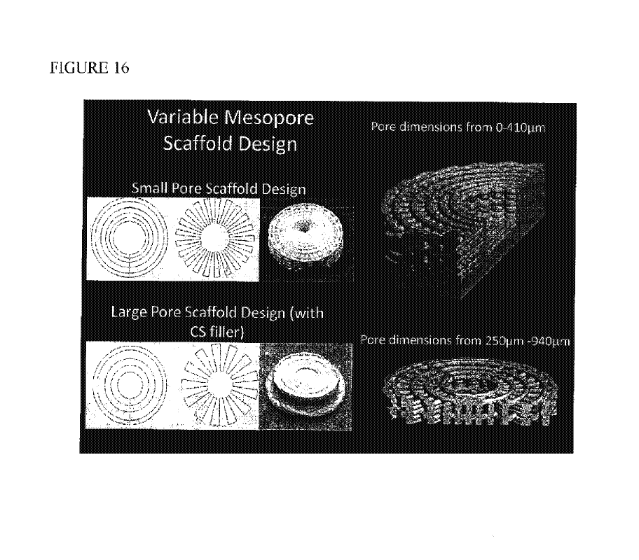

FIG. 17 depicts a scaffold architectural design including its relation to the surrounding bone and demonstrating the different forces acting on the scaffold during implantation including the pushing force from the skin (top) and the pushing force from the dura (bottom). The cap of the scaffold and dural side of the scaffold are indicated with arrows.

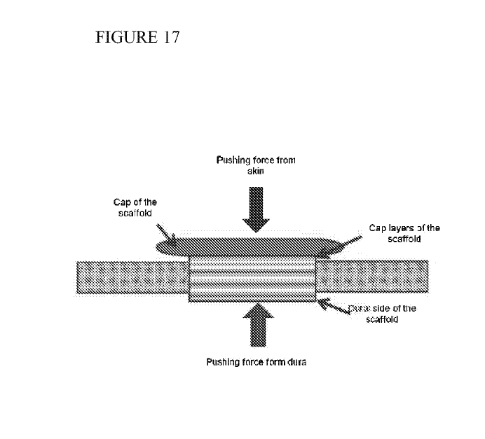

FIG. 18 depicts a computer generated scaffold design including a front view (A,C), a back view (B) and a side view (D).

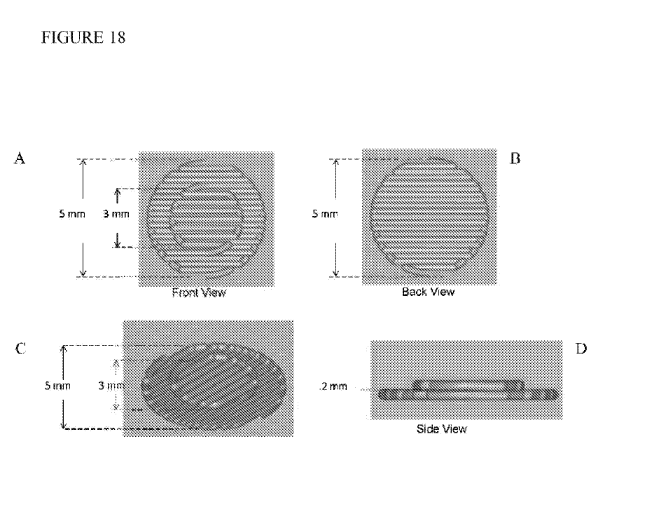

FIG. 19 represents a three dimensional Robocast Printer used to produce scaffolds as described herein.

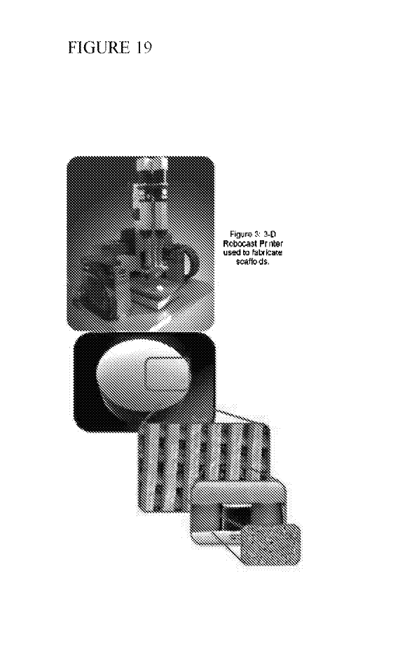

FIG. 20 depicts the timeline for the in vivo studies reported herein.

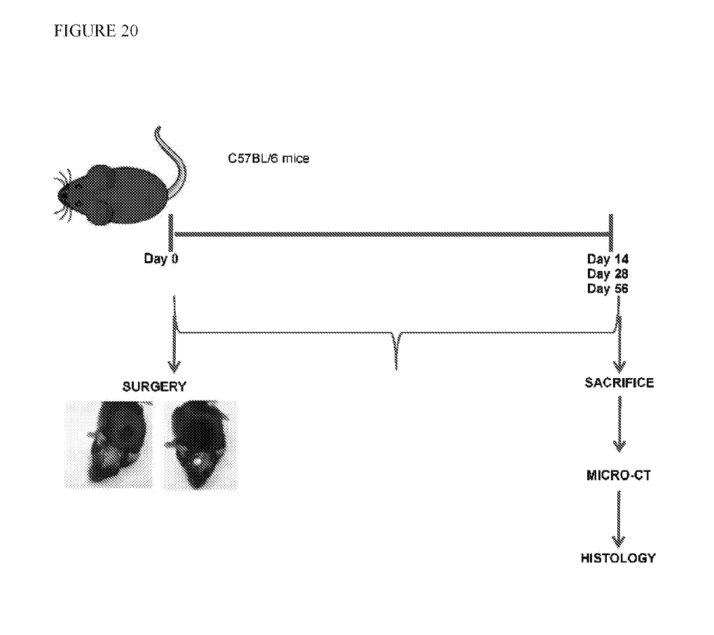

FIG. 21 provides SEM images of a 15/85% HA/b-TCP taken on a Hitachi S3500N at 5.0 kV and 3000×. (A) Green state with materials calcined to 800° C.; (B) Green state with materials calcined to 975° C.; (C, D) Rods sintered to 900° C. with materials calcined at 800° C. and 975° C., respectively; (E, F) Material sintered to 1100° C. and with initial materials prepared at 800° C. and 975° C., accordingly; (G, H) 15/85% material sintered to 1250° C. and calcination at 800° C. and 975° C., respectively.

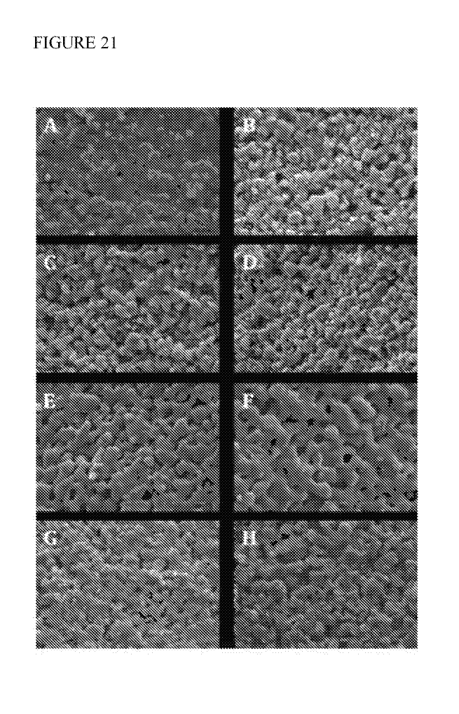

FIG. 22 represents the percent shrinkage mean±95% confidence interval for the different groups. The number of asterisks depicts statistically homogeneous groups.

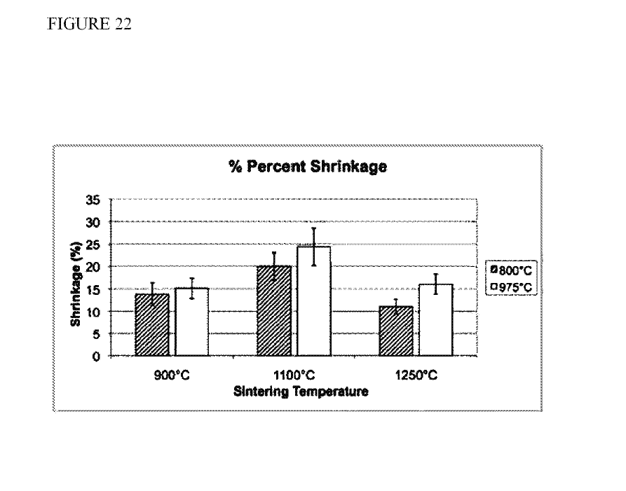

FIG. 23 represents the percent porosity mean±95% confidence interval for the different groups. The number of asterisks depicts statistically homogeneous groups.

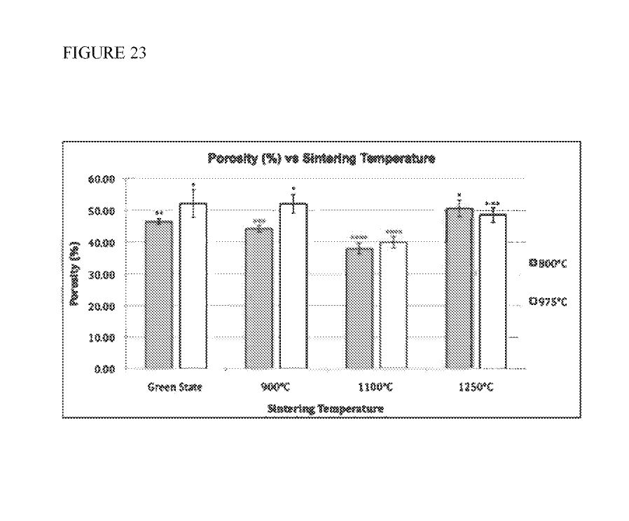

FIG. 24 provides an XRD spectrum that is a comparison between the two basic components of the ink. The yellow line is an XRD of raw hydroxyapatite. The black line represents the raw b-TCP.

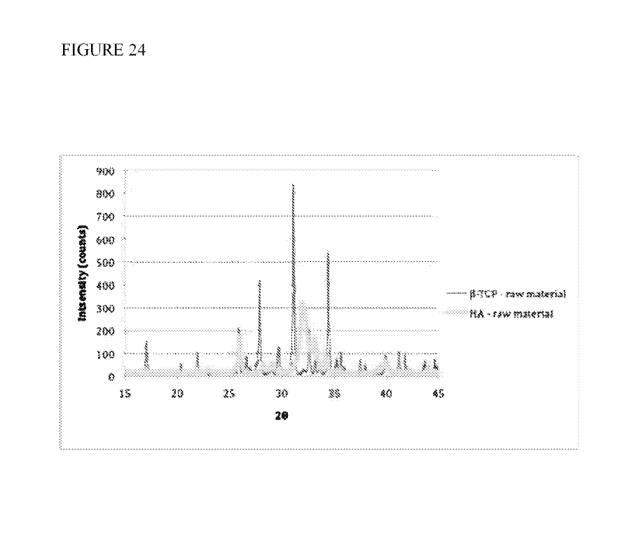

FIG. 25 provides an XRD spectrum for samples prepared with material calcined at 975° C. with respect to the different sintering stages. The green line is the green state, red line represents material sintered to 900° C., orange line represents sintering to 1100° C., and blue represents sintering to 1250° C. The rectangle shape points out the lack of HA triplet peaks.

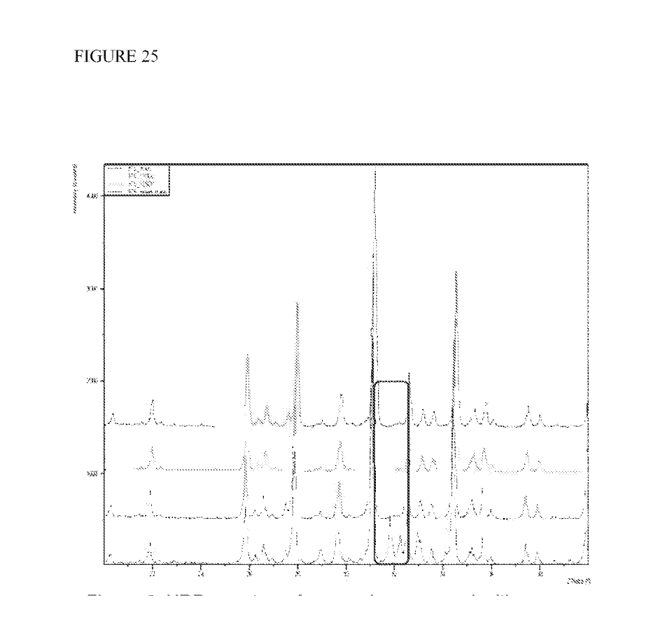

FIG. 26 provides an FT-IR spectra for (A) 100% HA sintered to 1250° C.; (B) 15/85% HA/b-TCP calcined at 800° C. and sintered to 1250° C.; and (C) 15/85% HA/b-TCP calacined at 975° C. and sintered to 1250° C.

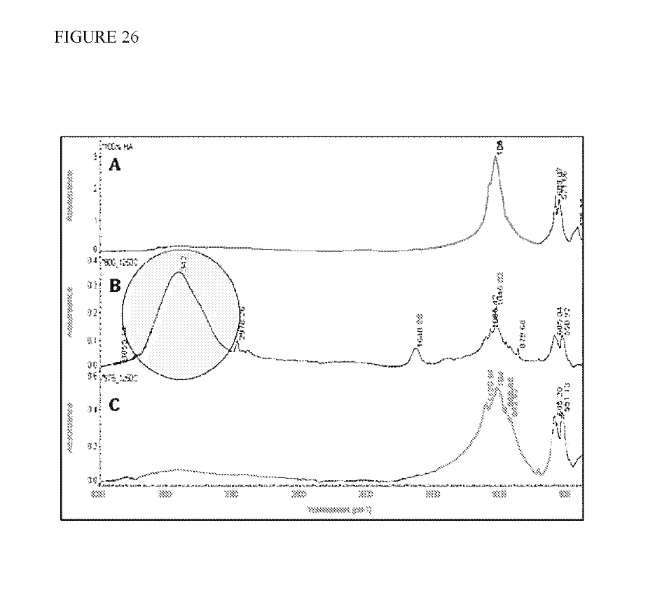

FIG. 27 is a graph showing the dipyridamole release in PBS. Collagen appears to have the more sustainable release.

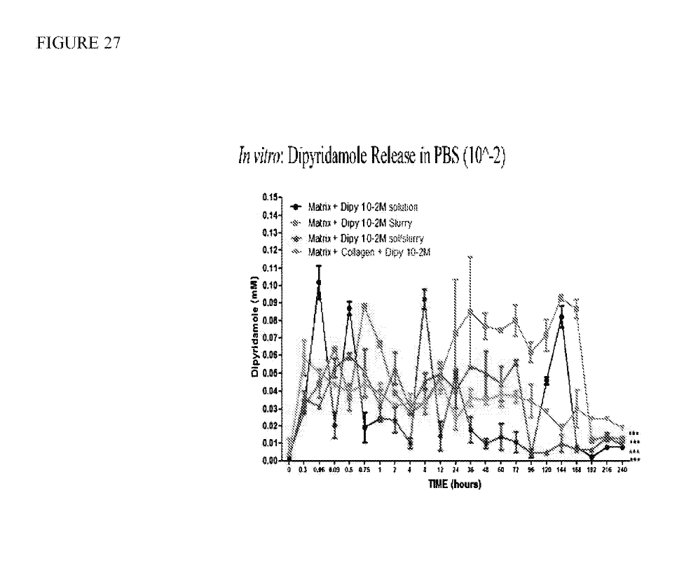

FIG. 28 is a graph showing new bone formation and remaining scaffold at 2 weeks after administration of BMP, dipyridamole and saline.

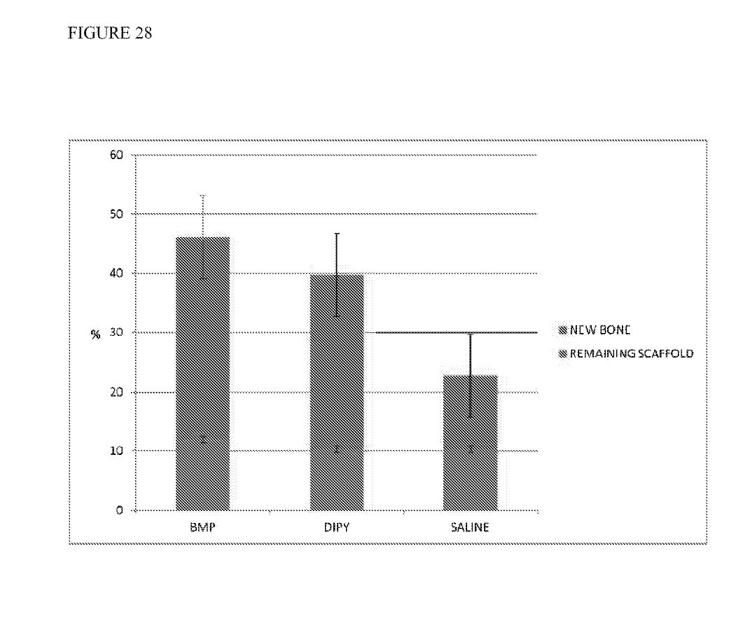

FIG. 29 (A, B) depicts histology 2 weeks after a scaffold containing BMP-2 is administered.

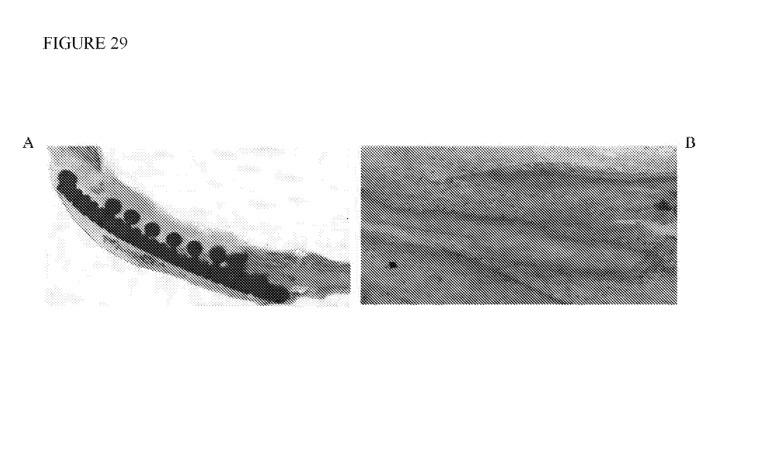

FIG. 30 (A, B) depicts histology 2 weeks after a scaffold containing dipyridamole is administered.

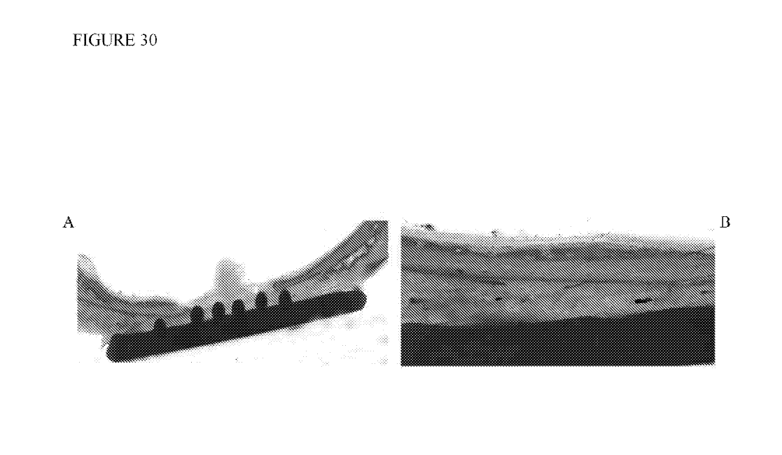

FIG. 31 (A, B) depicts histology 2 weeks after a scaffold containing PBS is administered.

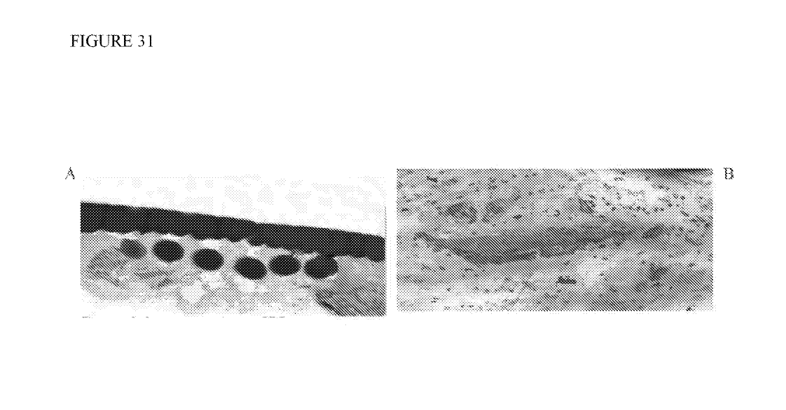

FIG. 32 (A, B) depicts histology 4 weeks after a scaffold containing BMP-2 is administered.

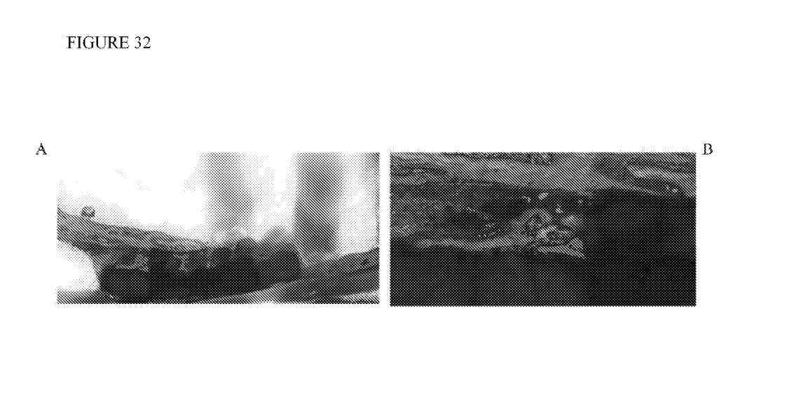

FIG. 33 (A, B) depicts histology 4 weeks after a scaffold containing dipyridamole is administered.

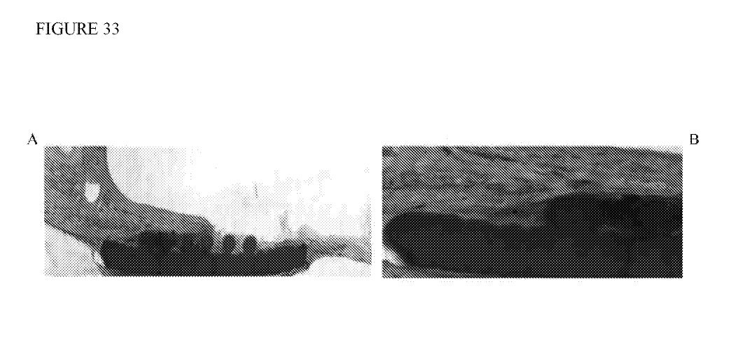

FIG. 34 (A, B) depicts histology 2 weeks after a scaffold containing PBS is administered.

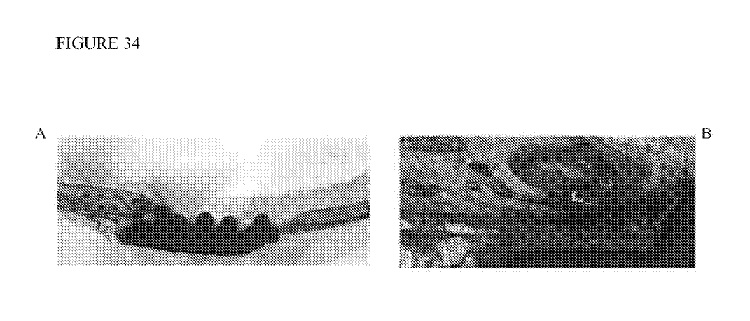

FIG. 35 is a graph showing new bone formation and remaining scaffold at 4 weeks after administration of BMP, dipyridamole and saline.

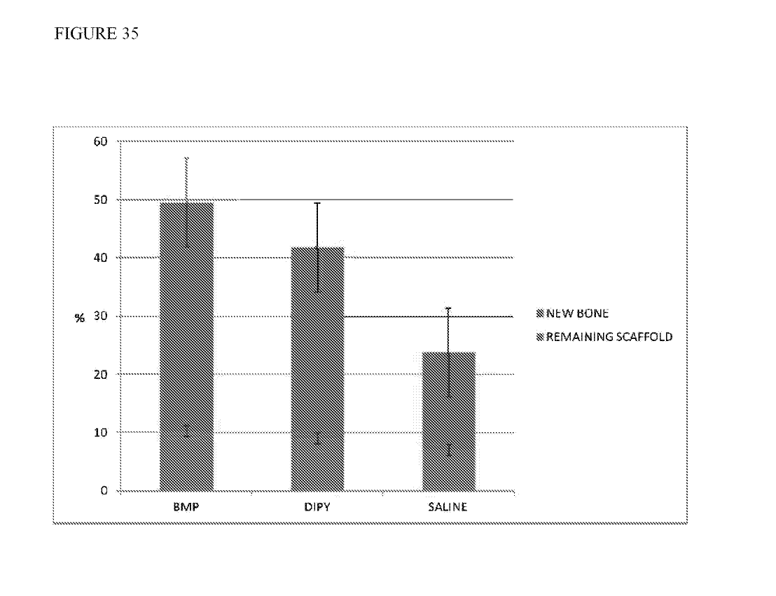

FIG. 36 is a graph showing new bone formation and remaining scaffold at 8 weeks after administration of BMP, dipyridamole and saline.

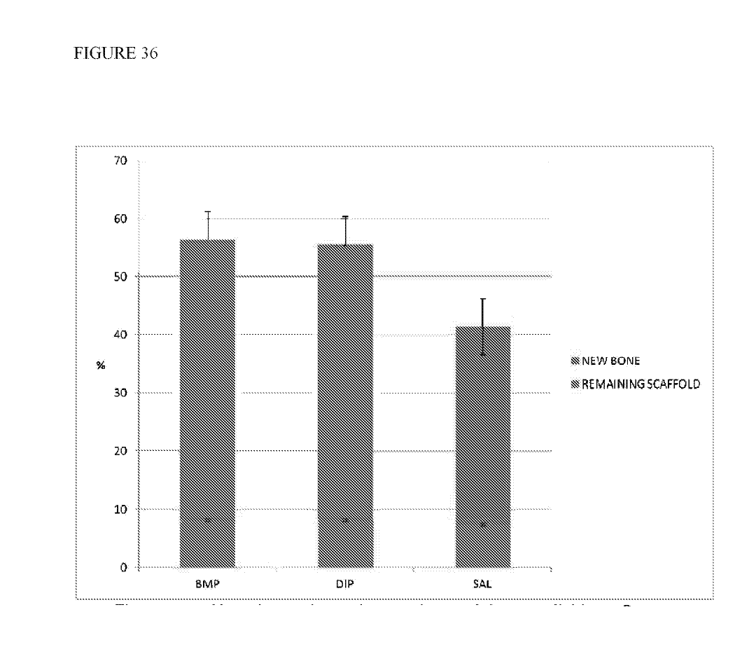

FIG. 37 (A, B) depicts histology 8 weeks after a scaffold containing BMP-2 is administered.

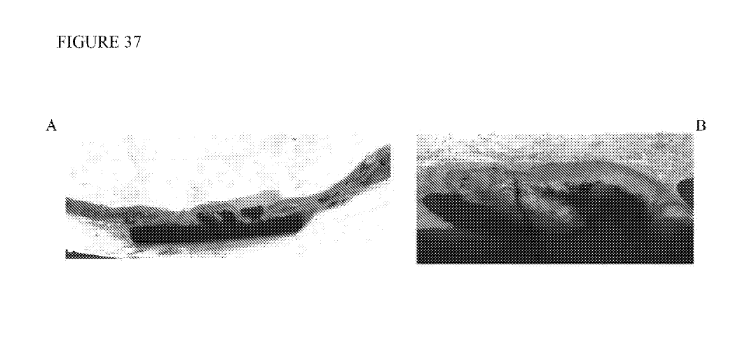

FIG. 38 (A, B) depicts histology 8 weeks after a scaffold containing dipyridamole is administered.

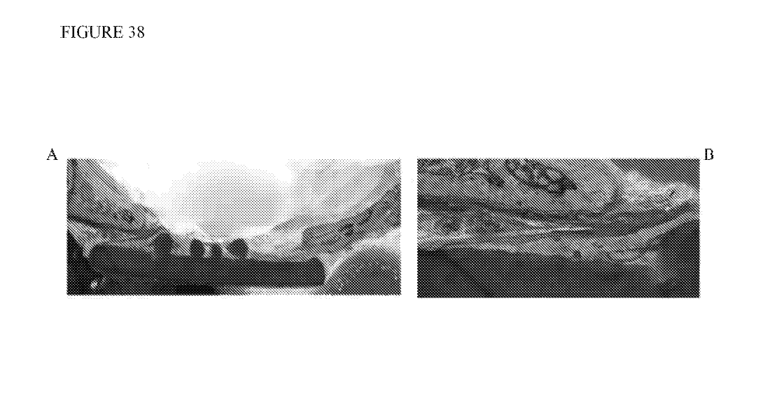

FIG. 39 (A, B) depicts histology 8 weeks after a scaffold containing PBS is administered.

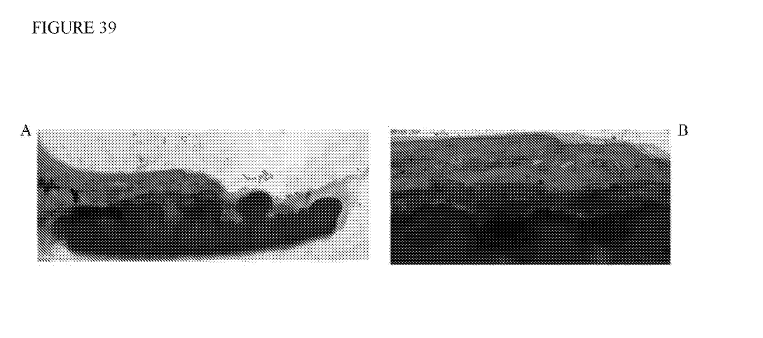

FIG. 40 is a graph showing in vivo bone formation. MicroCT was performed in adenosine A_{2A} knockout (A2AKO) mice and βTCP-HA scaffolds were coated with collagen and embedded in saline, CGS21680 (10 mM), Dipyridamole (100 μM) or BMP2 (200 ng). The graph shows the results obtained 4 weeks post-surgery. The remaining scaffold is shown in blue, and the new bone formation is shown in red. New bone formed on the dipyridamole coated surfaces did not differ from that induced in saline- or CGS21680-coated surfaces. However, resorption of scaffolds was greater in the saline and CGS21680-treated scaffolds.

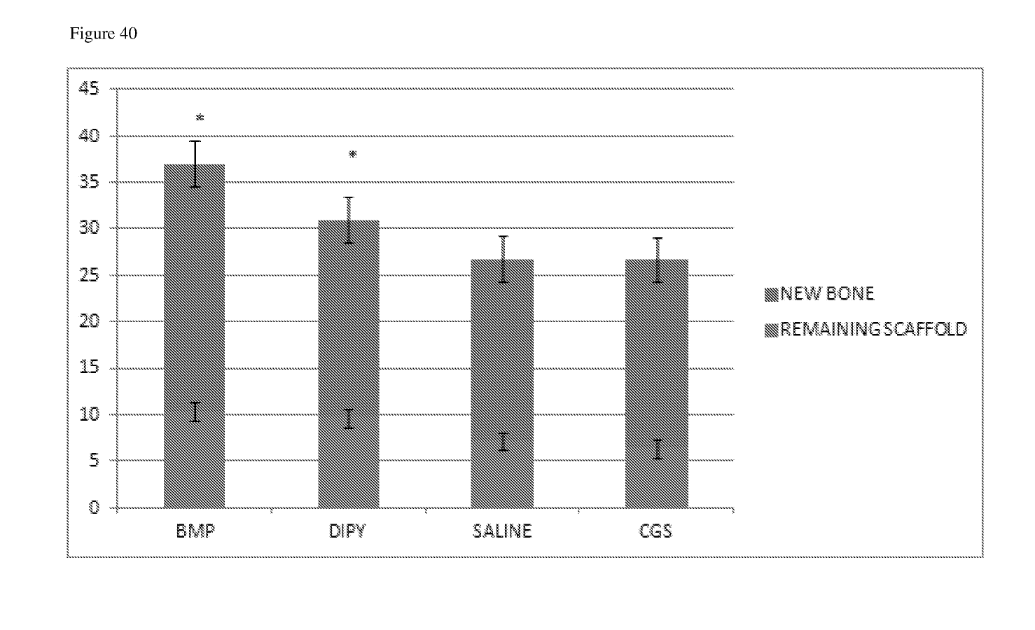

### DETAILED DESCRIPTION OF THE INVENTION

Multiphasic, three-dimensionally printed, tissue repair device (M3DRD) scaffolds may be used to replace current bone grafting techniques and bone graft substitutes, all of which have serious drawbacks and cannot be produced in the complex designs and shapes necessary for repair of complex bone defects. M3DRDs can be custom produced for complex grafting applications for craniofacial and orthopedic bone repair.

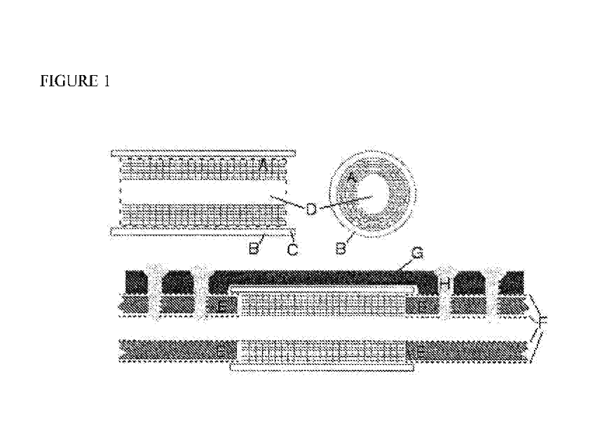

The multiphasic, three-dimensionally printed, tissue repair device (M3DRD) is a device beginning with at least one component, and possibly comprising three or more components (FIG. 1). The main components are (1) the scaffold, (2) the temporary filler/carrier material, and (3) a bioactive molecule/drug contained in the filler/carrier.

### The Scaffold

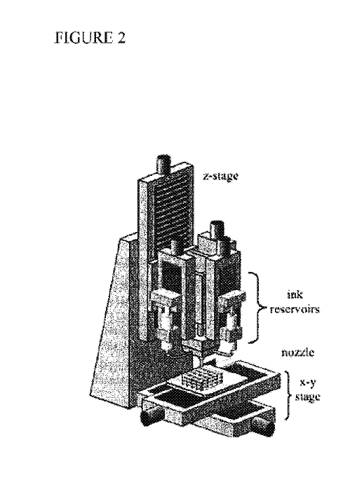

The core of the M3DRD is a three-dimensional scaffold that may be produced using a 3-D printing technique referred to as robotic deposition or direct write (DW) technology (See, FIG. 2). This technique uses a computer controlled printing process and colloidal inks to form three-dimensional structures. These structures can form on the self components or can be custom formed for filling individual bone defects from tomographic data (X-ray, sonographic or MRI).

Ink fabrication and the printing system itself are described in more detail in other references, but basically the system uses water-based rheologically controlled inks that become solid as they leave the print nozzle. These inks consist of finely controlled ceramic particles in a water-based slurry containing organic chemicals that control the handling characteristics of the colloidal ink. This allows 3-D lattice-like structures to be printed, in layers, without or with minimal sag of unsupported structural elements.

Using this system, the elements of the first layer may be printed by forcing the ink through a small (˜50-400 μm diameter) nozzle onto a support plate, using the x and y coordinate control system of an x-y-z control gantry system. Then the z control system is used to move the nozzle up slightly less than 1 nozzle diameter. Then the next layer is printed over the first layer. This is continued layer-by-layer until the entire 3-D structure is finished.

The entire structure may be printed in an oil bath to prevent drying. The system may have 3 nozzles and ink reservoirs so that up to three materials can be used to print a single structure. Fugitive inks, inks consisting entirely of material that burn off during firing, may also be used as part of the printing process. These can be used to print support structures for complex parts requiring temporary supports.

The resulting structures are then removed from the oil bath, dried, and fired in a programmable furnace to produce the final ceramic structure. Firing is currently done at approximately 1100° C. for about four hours, which substantially burns off the organic components, sintering the ceramic particles together into a solid structure. This may cause a small amount of predictable shrinkage that can be calculated into the printing process to produce precise and predictable structures.

The print nozzles may be routinely cylindrical producing cylindrical rod printed structures. However, nozzles may be made that are shaped to produce non-cylindrical structures or structures with surface striations of sizes designed to control cell migration, growth, and differentiation based on our earlier surface modification patents. (See, U.S. Pat. No. 6,419,491)

### Composition

Calcium phosphate base scaffolds were made from inks based upon permanent, remodelable (through bone remodeling processes), or soluble materials, or some combination of these. Some promising materials at this time are hydroxyapatite (HA) ceramics, tricalcium phosphate ceramics (TCP), and biphasic ceramics (HA/TCP) having a combination of the two materials. The HA materials produce permanent or very long-lasting scaffolds (depending on firing temperatures), the HA/TCP combinations may be varied with high HA percentages producing long-lasting scaffolds, and ˜99% TCP/1% HA scaffolds have been used to produce scaffolds that have been shown to remodel significantly through osteoclastic activity. Some such scaffolds contain approximately 3 mm thick, 11 mm diameter porous disks, with varying pore structures in different regions of the disk, and about a 0.5 mm thick solid cap structure of about 12 mm diameter. These have been inserted into 11 mm diameter trephine holes in rabbit parietal (skull) bones to test the bone and soft tissue response. It was demonstrated that these scaffolds can effectively be produced to have combinations of solid shell components to restrict fibrous tissue infiltration, and internal lattice structures with 270 μm diameter elements (this diameter can be varied using nozzle size) and pores (mesopores) ranging in size from less than 100 μm to 1000 μm in largest dimension. These constructs, with pores and strut sizes above the micron scale and below millimeter scale are referred to as mesostructures. The lattice structures, because of the HA and TCP composition, promote osteoconduction of new bone into the scaffolds. By adding small organic particles to the inks, microporous (on a submicron to −20 μm pore size) scaffold components can also be produced. These can be designed to attach fibrous connective tissue. Using these combinations of solid layers, various size open-weave mesopore lattices, microstructured lattice elements, and microporous lattice elements, complex structures can be designed and fabricated to conduct the ingrowth and formation of bone, marrow tissue, fibrous tissue, and blood vessels. An example of a scaffold for long bone regeneration is shown in FIG. 1. Since the DW system can print more than one material in a scaffold, it is feasible to print scaffolds with permanent HA components as well as remodelable TCP elements. This may be applicable in orthopedic applications where long-term strength of the scaffold is necessary.

### The Scaffold Filler/Carrier Material and Bioactive Factors

This filler/carrier component has a cement, polymer, or organic/natural hydrogel-based material that may be used to infiltrate the scaffold to produce a solid or nearly solid (if the filler is microporous) composite structure. This filler/carrier material may be soluble at some known or controlled rate, provide the scaffold with greater initial mechanical strength and stability, and then dissolve to allow and/or stimulate bone or soft tissue ingrowth (depending on the application and design). The filler/carrier may dissolve from the outside of the scaffold inward to its center, allowing the composite to become porous, as the scaffold component is exposed, and as tissue and blood vessels grow in from the surrounding tissue. This component may also protect the internal portion of the scaffold from the formation of a blood clot that may normally form there during early healing. This blood clot may become infected in oral and craniofacial sites where these sites are often non-sterile, or may become a granulation/fibrous tissue or necrotic either of which can impede bone ingrowth. The filler/carrier material may inherently stimulate tissue formation, or it may contain incorporated drugs, growth factors, cytokines, or antibiotics.

Some exemplary filler/carrier materials are calcium sulfate (plaster of paris), timed release calcium sulfate (a slow-dissolution version of calcium sulfate), and chitosan, a derivative of chitin, a biologically-derived polysaccharide, that can be used as a coating or hydrogel filler. Other materials, such as resorbable polymers like pol(L-lactic acid) (PLLA), may be used as filler/carrier materials, but alternatively these may be used as a coating material for the scaffold rather than filler. As such, they can still strengthen the scaffold and act as release materials, but may not be utilized to fill the scaffold and make it a solid structure.

Calcium sulfate was used as a filler and as a drug carrier material, where it was found to enhance mechanical properties of the structures, release biologically active agents in a predictable way, and not interfere with bone formation. Bioactive molecules investigated using this carrier include recombinant Platelet derived Growth Factor (PDGF) and Bone Morphogenetic Protein (BMP).

### Using Scaffold Mesostructure to Control Scaffold Mechanical Characteristics, Bone Characteristics, and Scaffold Remodeling

It is possible to design and produce scaffolds with mechanical properties suitable for use in craniofacial bone repair, and which, with some external support, are appropriate for orthopaedic repair. Scaffold mesostructure may also be used to control the structural characteristics and density of bone that is conducted into the scaffolds. Using a rabbit 11 mm diameter trephine defect as a model, three different design scaffolds were produced to fill the defects and examine bone regeneration. All scaffolds were produced of the same material, 99% TCP/1% HA ceramic, and were made of the same sized printed struts that were 270 μm in diameter. All scaffolds were also filled with medical grade calcium sulfate, and started as solid structures. Mesostructure was varied using strut spacing in the layers of the scaffold (x and y directions) and by stacking struts in the z direction. One type of scaffold that contained three strut spacings that produced open pores that were referred to (in the x and y directions) as 250×250 μm, 250×400 μm, and 400×400 μm size pores was produced (these dimensions are approximate). “Z” spacing was slightly less than one strut in height, or 230 μm. As measured by microcomputed tomography, these three zones had scaffold volume percentages of 46, 56, and 70%.

Two scaffolds were produced that had continuously variable porosity produced using radial struts alternating with concentric rings of different spacings. One scaffold had layers of 1 z and 2 z spacing and ring-shaped regions with scaffold volumes ranging from 55 to 94%. The other scaffold had 3 z spacing and regions ranging from 41 to 56% volume. Thus, a range of scaffold volumes were tested ranging from 41 to 94% scaffold. In all scaffolds, bone was capable of consistently growing to the center of the defect (across 5.5 mm distance) by 8 weeks.

This extent of consistent bone infiltration has not been observed in other osteoconductive scaffolds, and is due to the size and organization of the scaffold elements in the scaffolds. By using many small struts, in the size range of bone trabeculae, to conduct ingrowth, and by organizing them in ways that conduct bone in straight lines across the defects, it is possible to optimize the process of osteoconduction. This process, referred to as “directed osteoconduction” is novel to this type of scaffold. In scaffolds with random pore organization, the process of directed osteoconduction is not observed, and there consistent growth across large defects takes longer to occur. With the structures described herein, bone volumes at 8 and 16 weeks ranged from 9 to 40% (8 weeks) and 10 to 56% (16 weeks). Bone volume was inversely related to scaffold volume. More open (lower scaffold volume) scaffolds showed more bone ingrowth, and bone increased over time. Scaffold remodeling ranged from 5% to 56%, with more remodeling being observed in more open scaffolds at later time periods. Higher volume scaffolds (with smaller pores) produced more compact, lamellar bone, with the combination of scaffold and bone showing very little soft tissue and resembling a cortex-like structure. In contrast, lower volume scaffolds (with larger pores) produced more porous, disorganized bone, with the combination of bone and scaffold resembling cancellous bone. The type of bone adjacent to the scaffold (cortical or cancellous) at least partially influenced the bone growing in the adjacent scaffold.

### Features of M3DRD Scaffolds

In all, this data shows that osteoconductive scaffolds with designed mesostructures can be made with mechanical properties suitable for a wide range of bone repair applications. These scaffolds can be used to regenerate bone across significant distances without the need for bone cell or stem cell augmentation. The observed rate of osteoconduction across large defects is due to “directed osteoconduction” based on the use of many small struts, in the size range of bone trabeculae, that are organized in straight arrays to conduct bone efficiently across large distances.

The scaffolds can also be used to control resulting bone density, structure, and scaffold remodeling rates. The M3DRD scaffolds can be designed so that they regenerate bone that microstructurally approximates or matches adjacent bone. That is, where cancellous bone is needed, it is possible to regenerate cancellous structure, and where cortical bone is needed, it is possible to regenerate that form as well. Additional features like solid cap layers may successfully prevent soft tissue ingrowth. The CS filler may temporarily enhance structural mechanical properties and not impede bone formation and prevent fibrous tissue ingrowth and infiltration by infection and allow angiogenesis to proceed.

The CS can also be used for controlled release of bioactive molecules such as adenosine receptor agonists or antagonists or molecules that increase the biological activity or amount of adenosine. Use of the DW printing system allows custom design and printing of complex mesostructures with micron scale accuracy. This allows both off-the-shelf printed structures as well as custom printed M3DRD scaffolds for repair of complex defects in patients, based on MRI or CT data. This technology has widespread application in the craniofacial and orthopedic bone repair/replacement fields.

### Exemplary Tissue Repair Device or Scaffold

Bone defects are currently filled by complex autogenous grafting procedures; or imperfect allogeneic or alloplastic treatments not designed for a specific site. Direct Write (DW) fabrication allows printing 3-D scaffolds composed of osteoconductive biomaterials, complex multicomponent biphasic (COMBI) calcium phosphate scaffolds that have the potential to be custom-fabricated to repair complex bone defects. Current literature still debates optimum and threshold pore requirements for bone regeneration. Scaffolds were tested in a critical-sized (unable to close on its own) in vivo model to study effects on bone density, extent of ingrowth, and bone/scaffold remodeling.

Scaffolds were designed with variable mesopore spacing in all (X, Y, and Z) planes. To vary pore sizes, two scaffold designs of layers of concentric circles, alternating with radial struts of 1, 2, or 3 overlapping layers in z height, were fabricated by DW from 15:85 HAP/β-TCP and sintered at 1100° C. A calcium sulfate temporary filler prevented soft tissue invasion and/or infection. Scaffolds were embedded in vivo in trephine defects. After 8-16 weeks, analysis of bone ingrowth and scaffold and bone remodeling was quantified by MicroCT (Scanco Medical) and scaffolds were embedded in polymethylmethacrylate (PMMA) then evaluated histologically with light microscope.

Scaffold volume was designed to vary by ring section. Bone volume was higher in the more open, less scaffold-dense areas. Pores ranged from around 100 to 940 microns. Bone grew into all varied height layers, but appeared to take longer to get through the largest pore sizes. Pores larger than 500 microns still filled with bone well contrary to previous literature findings.

Particular scaffolds used demonstrated that three dimensional printed calcium phosphate scaffolds are capable of growing bone across at least 11 mm voids in 8 weeks. Bone can grow into pores as large as 940 μm and as small as 20 μm. Bone morphology can be trabecular-like or cortical-like depending on scaffold design. The scaffolds may be designed with regionally different biological and mechanical properties for a wide range of clinical applications.

The present invention demonstrates that a matrix containing either an adenosine A₁ receptor antagonist or an A_{2A} receptor stimulus at the site of a bone defect dramatically increases repair of the defect with new bone. Moreover, increase of endogenous adenosine levels at the site of the bone defect by application of dipyridamole, an agent that has been in clinical use for nearly 50 years to diminish platelet function and to induce coronary vasodilation by increasing endogenous adenosine levels, similarly promotes new bone growth at the site of a bone defect.

Promotion of local bone growth is critical for rapid healing of bone defects following trauma or invention. Similarly, agents that promote bone growth are commonly applied in a gel at the site of spinal fusion and other similar procedures. Currently BMPs are the principal stimuli for bone growth during spinal fusion or at sites of trauma. However, recent studies indicate that use of BMPs to promote bone growth during spinal fusion is associated with a significant increase in the risk for developing cancer. This understanding presents a novel opportunity for developing new agents useful for promoting bone growth to repair bone defects or stimulate formation of new bone during such procedures as spinal fusion.

The methods and structures described promote bone repair following trauma or bone growth after spinal fusion or similar surgeries. Application in a structure as described herein by a gel or matrix to bone defects of either A₁ receptor antagonists or A_{2A} agonists or dipyridamole to bone defects are provided herein.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth in their entirety.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference I their entireties.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

### DEFINITIONS

The terms used herein have the meanings recognized and known to those of skill in the art, however, for convenience and completeness, particular terms and their meanings are set forth below.

“Agent” refers to all materials that may be used to prepare pharmaceutical and diagnostic compositions, or that may be compounds such as small synthetic or naturally derived organic compounds, nucleic acids, polypeptides, antibodies, fragments, isoforms, variants, or other materials that may be used independently for such purposes, all in accordance with the present invention.

By “agonist” is meant a substance that binds to a specific receptor and triggers a response in a cell. It mimics the action of an endogenous ligand (such as hormone or neurotransmitter) that binds to the same receptor. A “full agonist” binds (has affinity for) and activates a receptor, displaying full efficacy at that receptor. One example of a drug that acts as a full agonist is isoproterenol which mimics the action of acetylcholine at β adrenoreceptors. A “partial agonist” (such as buspirone, aripiprazole, buprenorphine, or norclozapine) also binds and activates a given receptor, but has only partial efficacy at the receptor relative to a full agonist. A “partial agonist” may also be considered a ligand that displays both agonistic and antagonistic effects—when both a full agonist and partial agonist are present, the partial agonist actually acts as a competitive antagonist, competing with the full agonist for receptor occupancy and producing a net decrease in the receptor activation observed with the full agonist alone. A “co-agonist” works with other co-agonists to produce the desired effect together. An antagonist blocks a receptor from activation by agonists. Receptors can be activated or inactivated either by endogenous (such as hormones and neurotransmitters) or exogenous (such as drugs) agonists and antagonists, resulting in stimulating or inhibiting a biological response. A ligand can concurrently behave as agonist and antagonist at the same receptor, depending on effector pathways.

The potency of an agonist is usually defined by its EC₅₀ value. This can be calculated for a given agonist by determining the concentration of agonist needed to elicit half of the maximum biological response of the agonist. Elucidating an EC₅₀ value is useful for comparing the potency of drugs with similar efficacies producing physiologically similar effects. The lower the EC₅₀, the greater the potency of the agonist and the lower the concentration of drug that is required to elicit a maximum biological response.

“Antagonist” refers to an agent that down-regulates (e.g., suppresses or inhibits) at least one bioactivity of a protein. An “antagonist” or an agent that “antagonizes” may be a compound which inhibits or decreases the interaction between a protein and another molecule, e.g., a target peptide or enzyme substrate. An antagonist may also be a compound that down-regulates expression of a gene or which reduces the amount of expressed protein present. Methods for assessing the ability of an agent to “antagonize” or “inhibit” an adenosine receptor are known to those skilled in the art.

“Analog” as used herein, refers to a chemical compound, a nucleotide, a protein, or a polypeptide that possesses similar or identical activity or function(s) as the chemical compounds, nucleotides, proteins or polypeptides having the desired activity and therapeutic effect of the present invention (e.g. to treat or prevent bone disease, or to modulate osteoclast differentiation), but need not necessarily comprise a compound that is similar or identical to those compounds of the preferred embodiment, or possess a structure that is similar or identical to the agents of the present invention.

“Derivative” refers to the chemical modification of molecules, either synthetic organic molecules or proteins, nucleic acids, or any class of small molecules such as fatty acids, or other small molecules that are prepared either synthetically or isolated from a natural source, such as a plant, that retain at least one function of the active parent molecule, but may be structurally different. Chemical modifications may include, for example, replacement of hydrogen by an alkyl, acyl, or amino group. It may also refer to chemically similar compounds which have been chemically altered to increase bioavailability, absorption, or to decrease toxicity. A derivative polypeptide is one modified by glycosylation, pegylation, or any similar process that retains at least one biological or immunological function of the polypeptide from which it was derived.

By “medical prosthetic device” or “prosthesis” is meant an artificial component, device or extension that replaces a portion or all of a body part whether the body part is entirely or partially missing. The term includes artificial limbs, breast prosthesis such as those implanted post-mastectomy, cochlear implants, corrective lenses, craniofacial prosthesis, dental/maxillofacial prosthetics such as those implanted to correct a cleft palate, dentures, dental restoration, facial prosthetics, hair prosthesis, neuroprosthetics, ocular prosthetics, ostomies such as colostomy, ileostomy and urostomy, penile prosthetics, replacement joints such as hips, knees and shoulders, simato prosthetics, prosthetic testis and transtibial prosthesis.

A “small molecule” refers to a molecule that has a molecular weight of less than 3 kilodaltons (kDa), preferably less than about 1.5 kilodaltons, more preferably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is normally an organic compound (or organic compound complexed with an inorganic compound (e.g., metal)) that has a molecular weight of less than 3 kilodaltons, and preferably less than 1.5 kilodaltons, and more preferably less than about 1 kDa.

“Diagnosis” or “screening” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

The concept of “combination therapy” is well exploited in current medical practice. Treatment of a pathology by combining two or more agents that target the same pathogen or biochemical pathway sometimes results in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect can be synergistic (the efficacy of the combination is greater than the sum of the effects of each drug given alone). As used herein, the term “combination therapy” means the two compounds can be delivered in a simultaneous manner, e.g. concurrently, or one of the compounds may be administered first, followed by the second agent, e.g sequentially. The desired result can be either a subjective relief of one or more symptoms or an objectively identifiable improvement in the recipient of the dosage.

“Differentiate” or “differentiation” as used herein, generally refers to the process by which precursor or progenitor cells differentiate into specific cell types. In the present invention, the term refers to the process by which pre-osteoblasts become osteoblasts or pre-osteoclasts become osteoclasts. Differentiated cells can be identified by their patterns of gene expression and cell surface protein expression. As used herein, the term “differentiate” refers to having a different character or function from the original type of tissues or cells. Thus, “differentiation” is the process or act of differentiating. The term “Osteoclast Differentiation” refers to the process whereby osteoclast precursors in the bone marrow become functional osteoclasts, and the term “Osteoblast Differentiation” refers to the process whereby osteoblast precursors in the bone marrow become functional osteoblasts.

“Modulation” or “modulates” or “modulating” refers to up regulation (i.e., activation or stimulation), down regulation (i.e., inhibition or suppression) of a response, or the two in combination or apart. As used herein, an adenosine receptor “modulator” or “modulating” compound or agent is a compound or agent that modulates at least one biological marker or biological activity characteristic of osteoclasts and bone formation. The term “modulating” as related to osteoclast differentiation, refers to the ability of a compound or agent to exert an effect on precursors to osteoclasts, or to alter the expression of at least one gene related to osteoclastogenesis. For example, expression of the following genes is modulated during osteoclastogenesis: DC-Stamp, tartrate resistant alkaline phosphatase (TRAP), cathepsin K, calcitonin receptor, and integrin.

As used herein, the term “candidate compound” or “test compound” or “agent” or “test agent” refers to any compound or molecule that is to be tested. As used herein, the terms, which are used interchangeably, refer to biological or chemical compounds such as simple or complex organic or inorganic molecules, peptides, proteins, oligonucleotides, polynucleotides, carbohydrates, or lipoproteins. A vast array of compounds can be synthesized, for example oligomers, such as oligopeptides and oligonucleotides, and synthetic organic compounds based on various core structures, and these are also included in the terms noted above. In addition, various natural sources can provide compounds for screening, such as plant or animal extracts, and the like. Compounds can be tested singly or in combination with one another. Agents or candidate compounds can be randomly selected or rationally selected or designed. As used herein, an agent or candidate compound is said to be “randomly selected” when the agent is chosen randomly without considering the specific interaction between the agent and the target compound or site. As used herein, an agent is said to be “rationally selected or designed”, when the agent is chosen on a nonrandom basis which takes into account the specific interaction between the agent and the target site and/or the conformation in connection with the agent's action.

“Treatment” or “treating” refers to therapy, prevention and prophylaxis and particularly refers to administering medicine or performing medical procedures on a patient, for either prophylaxis (prevention) or to cure or reduce the extent of or likelihood of occurrence of the infirmity or malady or condition or event. In the present invention, the treatments using the agents described may be provided to stimulate or promote bone regeneration, to slow or halt bone loss, or to increase the amount or quality of bone density. Most preferably, the treating is for the purpose of stimulating or promoting bone regeneration or reducing or diminishing bone resorption. Treating as used herein also means administering the compounds for increasing bone density or for modulating osteoblastogenesis or osteoclastogenesis in individuals.

“Subject” or “patient” refers to a mammal, preferably a human, in need of treatment for a condition, disorder or disease.

“Osteoclastogenesis” refers to osteoclast generation, which is a multi-step process that can be reproduced in vitro. Earlier in vitro osteoclastogenesis systems used mixtures of stromal or osteoblastic cells together with osteoclast precursors from bone marrow (Suda, et al., (1997) Methods Enzymol. 282, 223-235; David et al., (1998) J. Bone Miner. Res. 13, 1730-1738). These systems utilized 1α, 25-dihydroxyvitamin D₃ to stimulate stromal/osteoblastic cells to produce factors that support osteoclast formation More recent models utilize bone marrow cells cultured with soluble forms of the cytokines M-CSF (macrophage-colony stimulating factor) and a soluble form of RANKL (receptor activator of nuclear factor KB ligand) (Lacey, et al., (1998) Cell 93, 165-176; Shevde et al., (2000) Proc. Natl. Acad. Sci. U.S.A. 97, 7829-7834). These two cytokines are now recognized as the major factors from stromal cells that support osteoclastogenesis (Takahashi, et al., (1999) Biochem. Biophys. Res. Commun. 256, 449-455). Thus, their addition to the culture medium overcomes the need for stromal cells.

“Osteoclast precursor” refers to a cell or cell structure, such as a pre-osteoclast, which is any cellular entity on the pathway of differentiation between a macrophage and a differentiated and functional osteoclast. The term osteoclast includes any osteoclast-like cell or cell structure which has differentiated fully or partially from a macrophage, and which has osteoclast character, including but not limited to positive staining for tartrate-resistant acid phosphatase (TRAP), but which is not a fully differentiated or functional osteoclast, including particularly aberrantly differentiated or non functional osteoclasts or pre-osteoclasts.

“Osteoclast culture” refers to any in vitro or ex vivo culture or system for the growth, differentiation and/or functional assessment of osteoclasts or osteoclast precursors, whether in the absence or presence of other cells or cell types, for instance, but not limited to, osteoblasts, macrophages, hematopoietic or stromal cells.

“Osteoclast function”, as used herein, refers to bone resorption and the processes required for bone resorption.

An “amount sufficient to inhibit osteoclast differentiation, formation or function” refers to the amount of the adenosine receptor agonist sufficient to block either the differentiation, the formation or the function of osteoclasts, more particularly, an amount ranging from about 0.1 nM to about 10 μM, or more preferentially from about 0.1 nM to about 5 μM, and most preferentially from about 0.1 nM to about 1 μM in vitro. In vivo amounts of an adenosine receptor agonist such as an adenosine A_{2A} receptor agonist sufficient to block either the differentiation, the formation or the function of osteoclasts may range from about 0.1 mg/Kg of body weight per day to about 200 mg/Kg of body weight per day in vivo, or more preferentially from about 1 mg/Kg to about 100 mg/Kg, and most preferentially from about 25 mg/Kg to about 50 mg/Kg of body weight per day in vivo. It is understood that the dose, when administered in vivo, may vary depending on the clinical circumstances, such as route of administration, age, weight and clinical status of the subject in which inhibition of osteoclast differentiation, formation or function is desired.

In a specific embodiment, the term “about” means within 20%, preferably within 10%, and more preferably within 5% or even within 1%.

An “effective amount” or a “therapeutically effective amount” is an amount sufficient to stimulate or promote bone regeneration or decrease or prevent the symptoms associated with the conditions disclosed herein, including bone loss or in a decrease in bone mass or density, such as that which occurs with medical prosthetic devices or other related conditions contemplated for therapy with the compositions of the present invention. For example, an “effective amount” for therapeutic uses is the amount of the composition comprising an active compound herein required to provide reversal or inhibition of bone loss or delay the onset of prosthetic device loosening, increase and/or accelerate bone growth into prosthetic devices, etc. Such effective amounts may be determined using routine optimization techniques and are dependent on the particular condition to be treated, the condition of the subject, the route of administration, the formulation, and the judgment of the practitioner and other factors evident to those skilled in the art. The dosage required for the compounds of the invention is that which induces a statistically significant difference in bone mass between treatment and control groups. This difference in bone mass or bone loss may be seen, for example, as at least 1-2%, or any clinically significant increase in bone mass or reduction in bone loss in the treatment group. Other measurements of clinically significant increases in healing may include, for example, an assay for the N-terminal propeptide of Type I collagen, tests for breaking strength and tension, breaking strength and torsion, 4-point bending, increased connectivity in bone biopsies and other biomechanical tests well known to those skilled in the art. General guidance for treatment regimens may be obtained from experiments carried out in vitro or in animal models of the disease of interest. The “effective amount” or “therapeutically effective amount” may range from about 1 mg/Kg to about 200 mg/Kg in vivo, or more preferentially from about 10 mg/Kg to about 100 mg/Kg, and most preferentially from about 25 mg/Kg to about 50 mg/Kg in vivo.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

Binding compounds can also be characterized by their effect on the activity of the target molecule. Thus, a “low activity” compound has an inhibitory concentration (IC₅₀) (for inhibitors or antagonists) or effective concentration (EC₅₀) (applicable to agonists) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured.

An individual “at risk” may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of a bone disease or low bone mass or density or enhanced susceptibility to bone resorption. An individual having one or more of these risk factors has a higher probability of developing bone resporption than an individual without these risk factors.

“Prophylactic” or “therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Timed release” or “sustained release” or “extended release” is a mechanism used for active pharmaceutical or biological agents over time in order to be released slower and steadier into the bloodstream or the tissue of interest while having the advantage of being taken or administered at less frequent intervals than immediate-release formulations of the same drug or agent. Most timed release drugs or agents are formulated so that the active ingredient is embedded in a matrix of insoluble substance(s) (various: some acrylics, even chitin) such that the dissolving drug or agent must find its way out through the holes in the matrix. Some drugs are enclosed in polymer-based tablets with a laser-drilled hole on one side and a porous membrane on the other side. Stomach acids push through the porous membrane, thereby pushing the drug out through the laser-drilled hole. In time, the entire drug dose releases into the system while the polymer container remains intact, to be excreted later through normal digestion. In some formulations, the drug or agent dissolves into the matrix, and the matrix physically swells to form a gel, allowing the drug to exit through the gel's outer surface. Micro-encapsulation is also regarded as a technology to produce complex dissolution profiles. Through coating an active pharmaceutical ingredient or agent around an inert core, and layering it with insoluble substances to form a microsphere you are able to obtain more consistent and replicable dissolution rates in a convenient format you can mix and match with other instant release pharmaceutical ingredients in to any two piece gelatin capsule. The compounds and agents of the present invention may be administered in sustained or timed release forms or from sustained or timed release delivery formulations or systems. A description of exemplary sustained release materials may be found in, for instance, Remington's Pharmaceutical Sciences.

### Adenosine

Adenosine, a potent endogenous physiological mediator, regulates a wide variety of physiological processes via interaction with one or more of four G protein-coupled receptors (A₁, A_{2A}, A_{2B}, and A₃), expressed on many cell types, including neutrophils, macrophages, fibroblasts, and endothelial cells. Because adenosine A_{2A} receptors inhibit the formation of giant cells from peripheral blood monocytes in vitro it was determined that adenosine, acting through one or another of these receptors, regulated the formation of osteoclasts.

In one embodiment, agents that interact with (e.g., bind to) and block, agonize or stimulate an adenosine receptor, in particular, A_{2A} (e.g., a functionally active fragment), are identified in a cell-based assay system. In accordance with this embodiment, cells expressing an adenosine receptor, a fragment of an adenosine receptor, an adenosine receptor related polypeptide, or a binding fragment thereof, are contacted with a candidate compound or a control compound and the ability of the candidate compound to interact with the receptor or fragment thereof is determined. Alternatively, the ability of a candidate compound to compete for binding with a known ligand or compound known to bind the receptor is measured. If desired, this assay may be used to screen a plurality (e.g. a library) of candidate compounds. The cell, for example, can be of prokaryotic origin (e.g., E. coli) or eukaryotic origin (e.g., yeast, insect or mammalian). Further, the cells can express the receptor endogenously or be genetically engineered to express the receptor, a binding fragment or a receptor fusion protein. In some embodiments, the receptor or fragment thereof, or the candidate compound is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detecting an interaction between the A_{2A} receptor and a candidate compound. The ability of the candidate compound to interact directly or indirectly with a receptor or binding fragment thereof or a fusion protein or to modulate the activity of the receptor can be determined by methods known to those of skill in the art. For example, the interaction or modulation by a candidate compound can be determined by flow cytometry, a scintillation assay, immunoprecipitation or western blot analysis, based on the present description, or by a competitive radioreceptor assay.

Selecting the compounds that interact with or bind to an adenosine receptor or otherwise agonize or stimulate or antagonize or inhibit the receptor may be performed in multiple ways. The compounds may first be chosen based on their structural and functional characteristics, using one of a number of approaches known in the art. For instance, homology modeling can be used to screen small molecule libraries in order to determine which molecules are candidates to interact with the receptor thereby selecting plausible targets. The compounds to be screened can include both natural and synthetic ligands. Furthermore, any desired compound may be examined for its ability to interact with or bind to the receptor.

Binding to or interaction with adenosine receptors may be determined by performing an assay such as, for example, a binding assay between a desired compound and an adenosine receptor. In one aspect, this is done by contacting said compound to an adenosine receptor and determining its dissociation rate. Numerous possibilities for performing binding assays are well known in the art. The indication of a compound's ability to bind to an adenosine receptor is determined, e.g., by a dissociation rate, and the correlation of binding activity and dissociation rates is well established in the art. For example, the assay may be performed by radio-labeling a reference compound, or other suitable radioactive marker, and incubating it with the cell bearing an adenosine receptor, in particular, an A₁ or A_{2A}. Test compounds are then added to these reactions in increasing concentrations. After optimal incubation, the reference compound and receptor complexes are separated, e.g., with chromatography columns, and evaluated for bound ¹²⁵I-labeled peptide with a gamma (γ) counter. The amount of the test compound necessary to inhibit 50% of the reference compound's binding is determined. These values are then normalized to the concentration of unlabeled reference compound's binding (relative inhibitory concentration (RIC)⁻¹=concentrationₜₑₛₜ/concentration_{reference}). A small RIC value indicates strong relative binding, whereas a large RIC value indicates weak relative binding. See, for example, Latek et al., Proc. Natl. Acad. Sci. USA, Vol. 97, No. 21, pp. 11460-11465, 2000. An adenosine receptor agonist mimic may be computationally evaluated and designed by means of a series of steps in which chemical groups or fragments are screened and selected for their ability to associate with the individual binding pockets or interface surfaces of the protein (e.g. the A_{2A} receptor). One skilled in the art may employ one of several methods to screen chemical groups or fragments for their ability to associate with the adenosine receptor. This process may begin by visual inspection of, for example, the protein/protein interfaces or the binding site on a computer screen based on the available crystal complex coordinates of the receptor, including a protein known to interact with selected fragments or chemical groups may then be positioned in a variety of orientations, or docked, at an individual surface of the receptor that participates in a protein/protein interface or in the binding pocket. Docking may be accomplished using software such as QUANTA and SYBYL, followed by energy minimization and molecular dynamics with standard molecular mechanics forcefields, such as CHARMM and AMBER (AMBER, version 4.0 (Kollman, University of California at San Francisco, copyright, 1994); QUANTA/CHARMM (Molecular Simulations, Inc., Burlington, Mass., copyright, 1994)). Specialized computer programs may also assist in the process of selecting fragments or chemical groups. These include: GRID (Goodford, 1985, J. Med. Chem. 28:849-857), available from Oxford University, Oxford, UK; MCSS (Miranker & Karplus, 1991, Proteins: Structure, Function and Genetics 11:29-34), available from Molecular Simulations, Burlington, Mass.; AUTODOCK (Goodsell & Olsen, 1990, Proteins: Structure, Function, and Genetics 8:195-202), available from Scripps Research Institute, La Jolla, Calif.; and DOCK (Kuntz et al., 1982, J. Mol. Biol. 161:269-288), available from University of California, San Francisco, Calif. Once suitable chemical groups or fragments that bind to an adenosine receptor have been selected, they can be assembled into a single compound or agonist. Assembly may proceed by visual inspection of the relationship of the fragments to each other in the three-dimensional image displayed on a computer screen in relation to the structure coordinates thereof. This would be followed by manual model building using software such as QUANTA or SYBYL. Useful programs to aid one of skill in the art in connecting the individual chemical groups or fragments include: CAVEAT (Bartlett et al., 1989, ‘CAVEAT: A Program to Facilitate the Structure-Derived Design of Biologically Active Molecules’. In Molecular Recognition in Chemical and Biological Problems′, Special Pub., Royal Chem. Soc. 78:182-196), available from the University of California, Berkeley, Calif.; 3D Database systems such as MACCS-3D (MDL Information Systems, San Leandro, Calif.). This area is reviewed in Martin, 1992, J. Med. Chem. 35:2145-2154); and HOOK (available from Molecular Simulations, Burlington, Mass.). Instead of proceeding to build an adenosine receptor agonist mimic, in a step-wise fashion one fragment or chemical group at a time, as described above, such compounds may be designed as a whole or ‘de novo’ using either an empty binding site or the surface of a protein that participates in protein/protein interactions or optionally including some portion(s) of a known activator(s). These methods include: LUDI (Bohm, J. Comp. Aid. Molec. Design 1992; 6:61-78), available from Molecular Simulations, Inc., San Diego, Calif.; LEGEND (Nishibata et al., 1991, Tetrahedron 47:8985), available from Molecular Simulations, Burlington, Mass.; and LeapFrog (available from Tripos, Inc., St. Louis, Mo.). Other molecular modeling techniques may also be employed in accordance with this invention. See, e.g., Cohen et al., J. Med. Chem. 1990; 33:883-894. See also, Navia & Murcko, Current Opinions in Structural Biology 1992; 2:202-210.

Once a compound has been designed by the above methods, the efficiency with which that compound may bind to or interact with the adenosine receptor protein may be tested and optimized by computational evaluation. Agonists may interact with the receptor in more than one conformation that is similar in overall binding energy. In those cases, the deformation energy of binding is taken to be the difference between the energy of the free compound and the average energy of the conformations observed when the inhibitor binds to the receptor protein.

A compound selected for binding to the adenosine receptor may be further computationally optimized so that in its bound state it would preferably lack repulsive electrostatic interaction with the target protein. Such non-complementary electrostatic interactions include repulsive charge-charge, dipole-dipole and charge-dipole interactions. Specifically, the sum of all electrostatic interactions between the compound and the receptor protein when the mimic is bound to it preferably make a neutral or favorable contribution to the enthalpy of binding. Specific computer software is available in the art to evaluate compound deformation energy and electrostatic interaction. Examples of programs designed for such uses include: Gaussian 92, revision C (Frisch, Gaussian, Inc., Pittsburgh, Pa. copyright 1992); AMBER, version 4.0 (Kollman, University of California at San Francisco, copyright 1994); QUANTA/CHARMM (Molecular Simulations, Inc., Burlington, Mass., copyright 1994); and Insight II/Discover (Biosym Technologies Inc., San Diego, Calif., copyright 1994). These programs may be implemented, for instance, using a computer workstation, as are well-known in the art. Other hardware systems and software packages will be known to those skilled in the art.

Once an adenosine receptor modulating compound, such as an agonist, has been optimally designed, for example as described above, substitutions may then be made in some of its atoms or chemical groups in order to improve or modify its binding properties, or its pharmaceutical properties such as stability or toxicity. Generally, initial substitutions are conservative, i.e., the replacement group will have approximately the same size, shape, hydrophobicity and charge as the original group. Substitutions known in the art to alter conformation should be avoided. Such altered chemical compounds may then be analyzed for efficiency of binding to the receptor by the same computer methods described in detail above.

### Candidate Compounds and Agents

Examples of agents, candidate compounds or test compounds include, but are not limited to, nucleic acids (e.g., DNA and RNA), carbohydrates, lipids, proteins, peptides, peptidomimetics, small molecules and other drugs. In one preferred aspect, agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 1997; 12:145; U.S. Pat. No. 5,738,996 and U.S. Pat. No. 5,807,683).

Phage display libraries may be used to screen potential ligands or adenosine receptor modulators. Their usefulness lies in the ability to screen, for example, a library displaying a large number of different compounds. For use of phage display libraries in a screening process, see, for instance, Kay et al., Methods, 240-246, 2001. An exemplary scheme for using phage display libraries to identify compounds that bind or interact with an adenosine receptor may be described as follows: initially, an aliquot of the library is introduced into microtiter plate wells that have previously been coated with target protein, e.g. A₁ or A_{2A} receptor. After incubation (e.g., 2 hours), the nonbinding phage are washed away, and the bound phage are recovered by denaturing or destroying the target with exposure to harsh conditions such as, for instance pH 2, but leaving the phage intact. After transferring the phage to another tube, the conditions are neutralized, followed by infection of bacteria with the phage and production of more phage particles. The amplified phage are then rescreened to complete one cycle of affinity selection. After three or more rounds of screening, the phage are plated out such that there are individual plaques that can be further analyzed. For example, the conformation of binding activity of affinity-purified phage for the adenosine A_{2A} receptor may be obtained by performing ELISAs. One skilled in the art can easily perform these experiments. In one aspect, an A₁ or A_{2A} receptor molecule used for any of the assays may be a recombinant A₁ or A_{2A} receptor protein, or an A₁ or A_{2A} fusion protein, an analog, derivative, or mimic thereof.

Examples of methods for the synthesis of molecular libraries can be found in the art, for example in DeWitt et al., Proc. Natl. Acad. Sci. USA 1993; 90: 6909; Erb et al., Proc. Natl. Acad. Sci. USA 1994; 91: 11422; Zuckermann et al., J. Med. Chem. 1994; 37: 2678; Cho et al., Science 1993; 261: 1303; Carrell et al., Angew. Chem. Int. Ed. Engl. 1994; 33: 2059; Carell et al., Angew. Chem. Int. Ed. Engl. 1994; 33: 2061; and Gallop et al., J. Med. Chem. 1994; 37: 1233.

Libraries of compounds may be presented, e.g., in solution (Houghten, Bio/Techniques 1992; 13: 412-421), or on beads (Lam, Nature 1991; 354: 82-84), chips (Fodor, Nature 1993; 364: 555-556), bacteria (U.S. Pat. No. 5,223,409), spores (U.S. Pat. Nos. 5,571,698; 5,403,484; and 5,223,409), plasmids (Cull et al., Proc. Natl. Acad. Sci. USA 1992; 89: 1865-1869) or phage (Scott and Smith, Science 1990; 249: 386-390; Devlin, Science 1990; 249: 404-406; Cwirla et al., Proc. Natl. Acad. Sci. USA 1990; 87: 6378-6382; and Felici, J. Mol. Biol. 1991; 222: 301-310).

The methods of screening compounds may also include the specific identification or characterization of such compounds, whose effect on bone resorption is determined by the methods described above. If the identity of the compound is known from the start of the experiment, no additional assays are needed to determine its identity. However, if the screening for compounds that modulate the adenosine A_{2A} receptor is done with a library of compounds, it may be necessary to perform additional tests to positively identify a compound that satisfies all required conditions of the screening process. There are multiple ways to determine the identity of the compound. One process involves mass spectrometry, for which various methods are available and known to the skilled artisan (e.g. the neogenesis website). Neogenesis' ALIS (automated ligand identification system) spectral search engine and data analysis software allow for a highly specific identification of a ligand structure based on the exact mass of the ligand. One skilled in the art can also readily perform mass spectrometry experiments to determine the identity of the compound.

Antibodies, including polyclonal and monoclonal antibodies, particularly anti-A_{2A} receptor antibodies and neutralizing antibodies may be useful as compounds to modulate osteoclast differentiation and/or function. These antibodies are available from such vendors as Upstate Biologicals, Santa Cruz, or they made be prepared using standard procedures for preparation of polyclonal or monoclonal antibodies known to those skilled in the art. Also, antibodies including both polyclonal and monoclonal antibodies, and drugs that modulate the activity of the adenosine receptor and/or its subunits may possess certain diagnostic applications and may for example, be utilized for the purpose of detecting and/or measuring conditions such as bone diseases, bone loss, or osteoclast differentiation and/or function. The adenosine receptor or its subunits may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or act as agonists for the activities of the A_{2A} receptor may be discovered or synthesized, and may be used in diagnostic and/or therapeutic protocols.

### DETAILED DESCRIPTION OF THE PREFERRED EMBODIMENTS

### Example 1

### BACKGROUND

Bone healing is a proliferative physiological process that is well established and efficient yet not always predictable. The use of a three-dimensional (3-D) printing technique referred to as direct-write fabrication (DW), with its ability to produce scaffolds that direct the repair of natural bone, may represent an optimal solution for the fabrication of bone repair in the craniofacial, and orthopaedic arenas. The Beta-Tri-Calcium Phosphate (b-TCP)/Hydroxyapatite(HA) shell and strut components provide mechanical strength, conduct bone throughout the scaffold directionally and remodel over time. β-TCP scaffold material was one of the first to be utilized in vivo due to its similar composition to mineral phase native bone. β-TCP scaffolds are strongly osteoinductive, osteoconductive and exert their effects via interaction with a2b 1 integrins on osteogenic cells and subsequent downstream activation of MAPK/ERK signaling pathways in these cells. Numerous surgical practices include the use of growth factors, such as bone morphogenetic factors (BMPs) to promote the regeneration of bone. However, there have been persistent concerns regarding the adverse events by these growth factors. Although most approaches to promoting bone formation have involved stimulating osteoblast differentiation and function, e.g. by application of BMPs, it is also important to understand whether stimulation of adenosine A_{2A} receptors, which inhibit osteoclast formation and minimally affect osteoblast differentiation or inhibition of A₁ receptors could affect bone regeneration in an in vivo model.

Adenosine is a potent physiologic regulator, whose levels in the extracellular space are tightly regulated at the level of production, catabolism and facilitated cellular uptake (via ent1) with subsequent phosphorylation to adenine nucleotides. Adenosine mediates its physiologic and pharmacologic effects via binding to one or more G protein-coupled receptors (A₁, A_{2A}, A_{2B} and A₃). Studies indicate that blockade of adenosine A₁ receptors or stimulation of A_{2A} receptors on osteoclast precursors block osteoclast differentiation and function. In vivo effects have also been reported as well. Other studies have reported that stimulation of A_{2B} receptors stimulates osteoblast differentiation and bone production, primarily in in vitro studies. More recently, novel translational uses for agents that act, directly or indirectly, at these receptors have been developed. An adenosine A₁ antagonist, an A_{2A} agonist or an agent that increases local adenosine levels by blocking cellular uptake of adenosine (dipyridamole) exponentially stimulates bone regeneration in a murine model of calvarial bone regeneration.

Using specialized three-dimensional printing technology combined with fillers and bioactive molecules such as dipyridamole, it is possible to design, characterize, and demonstrate the efficacy of synthetic, off-the-shelf and custom fabricated, 3-D scaffolds for repair of bone defects. With a drug, such as dipyridamole, the combination of drug-scaffolds successfully regenerates bone over critical sized bone defects in an in vivo model.

### Materials and Methods

HA/β-TCP characterization, scaffold development and fabrication via direct-write printing technique: 85% b-TCP:15% HA ink was characterized using Micro-CT, SEM, X-Ray Diffraction, and ICP. 3-D Robocad software was used to design scaffolds and 3-D Printing Robocast was used to produce scaffolds. Scaffolds were sintered at 1100° C. for 4 hours. Post-sintering resulted in ˜95% b-TCP: 5% HA.

In vitro: The 3-D scaffold composition/calcium sulfate/collagen/dipyridamole combinations will be tested in vitro to determine dipyridamole release in PBS (10⁻²).

In vivo testing in mice: The 3-D scaffolds will be evaluated in a murine model using a 3 mm calvaria defect model. Scaffold composition/collagen/dipyridamole combinations will be evaluated at 2, 4 and 8 weeks.

### DISCUSSION

Targeting osteoblasts and osteoclasts via appropriate adenosine receptor blockade or stimulation leads to increased bone regeneration in a murine model. The mechanism by which adenosine receptor-mediated suppression of osteoclast function/accumulation stimulates bone regeneration may involve suppression of Semaphorin 4D permitting increased osteoblast formation of bone. Micro-CT and histology results show that the delivery of dipyridamole in the 3D ceramic scaffolds promotes bone formation as effectively as BMP-2 in vivo. Different inks can be used to fabricate different regions of the scaffold, depending on anticipated mechanical and remodeling requirements. Scaffolds may be filled with different component materials that can be released at different times. For instance, a certain portion of the long bone replacement segment may be filled with more concentrated dipyridamole or bone stimulating growth factor, designed to be released at a later time, than the regions adjacent to existing bone. Thus, this allows a specific, timed release of stimulating factors. Altering the outer cap, strut size and interconnectivity of the scaffold may also offer several potential advantages such as, for instance, continuous supply of nutrients, greater cellular and tissue ingrowth, and enhanced revascularization. Thus, these factors may result in better bone remodeling.

While the present invention has been set forth in terms of a specific embodiment or embodiments, it will be understood that the present scaffolds and methods herein disclosed may be modified or altered by those skilled in the art to other configurations. Accordingly, the invention is to be broadly construed and limited only by the scope and spirit of the claims appended hereto.

What is claimed is:

1. A tissue repair device or scaffold having a porous bone ingrowth structure containing interconnected struts surrounded by a microporous shell wherein the tissue repair device or scaffold contains therein or thereon a therapeutically effective amount of an adenosine receptor agonist, an adenosine receptor antagonist, or an agent that upregulates, increases the amount of or increases the biological activity of adenosine or an analog or derivative thereof.

2. A tissue repair device or scaffold according to claim 1 wherein the adenosine receptor agonist is an adenosine A_{2A} or adenosine A_{2B} receptor agonist.

3. A tissue repair device or scaffold according to claim 1 wherein the adenosine receptor antagonist is an adenosine A₁ receptor antagonist.

4. A tissue repair device or scaffold according to claim 1 wherein the agent that upregulates, increases the amount of or increases the biological activity of adenosine is dipyridamole.

5. A tissue repair device or scaffold according to claim 1 wherein the adenosine receptor agonist, an adenosine receptor antagonist, or an agent that upregulates, increases the amount of or increases the biological activity of adenosine or an analog or derivative thereof is provided in a sustained release formulation.

6. A tissue repair device or scaffold according to claim 1 wherein the microporous shell is extended as a guide to stabilize the tissue repair device or scaffold between one or more ends of bone.

7. The tissue repair device or scaffold according to claim 1 wherein the porous ingrowth structure is infiltrated with a soluble filler or carrier.

8. The tissue repair device or scaffold according to claim 1 further comprising a soluble filler wherein the soluble filler or carrier is infiltrated with one or more of an antibiotic, a growth factor, a differentiation factor, a cytokine, a drug, or a combination thereof.

9. The tissue repair device or scaffold according to claim 1 wherein the struts are from about 100-350 μm diameter.

10. The tissue repair device or scaffold according to claim 1 wherein the struts are within about 2× or substantially the same diameter as bone trabeculae.

11. The tissue repair device or scaffold according to claim 1 wherein one or more struts are separated longitudinally by a space of at least 500 μm.

12. The tissue repair device or scaffold according to claim 1 being porous and comprising mesopores present in a size generally more than about 20 μm diameter.

13. The tissue repair device or scaffold according to claim 1 wherein the struts are arranged in a substantially linear arrangement.

14. The tissue repair device or scaffold according to claim 1 being resorbable so that after about 8 weeks presence in vivo, at least about 25% of the tissue repair device or scaffold is resorbed.

15. The tissue repair device or scaffold according to claim 1 being at least about 50% porous.

16. The tissue repair device or scaffold according to claim 1 being operable to encourage and provide bone growth such that after about 8 weeks presence in vivo, at least about 25% of the tissue repair device or scaffold is replaced by bone.

17. The tissue repair device or scaffold according to claim 1 comprising micropores or nanopores having a diameter of about 0.1-1 μm.

18. The tissue repair device or scaffold according to claim 17 wherein one or more micropores or nanopores are infiltrated with solubilized collagen.

19. The tissue repair device or scaffold according to claim 1 produced by a three dimensional printing method.

20. A method for promoting bone growth or treating bone fracture, defect or deficiency comprising providing a tissue repair device or scaffold having a porous bone ingrowth structure containing interconnected struts surrounded by a microporous shell according to claim 1 in vivo to a region featuring a bone deficiency, fracture or void.

21. A method for producing a tissue repair device or scaffold useful for promoting bone growth or treating bone fracture, defect or deficiency having a porous bone ingrowth region containing interconnected struts surrounded by a microporous shell, comprising:
(a) providing microporous shell that may function to attach but limit soft tissue ingrowth,
(b) infiltrating the porous ingrowth structure with a soluble filler or carrier; and
(c) providing a therapeutically effective amount of an adenosine receptor agonist, an adenosine receptor antagonist, or an agent that upregulates, increases the amount of or increases the biological activity of adenosine or an analog or derivative thereof.

22. A method according to claim 21 further comprising (d) infiltrating the porous ingrowth structure with one or more of an antibiotic, a growth factor, a differentiation factor, a cytokine, a drug, or a combination of these agents.
